(19) United States
(12) Patent Application Publication
(10) Pub. No.: US 2025/0375293 A1
(43) Pub. Date: Dec. 11, 2025

(54) TISSUE PERFORATION ASSEMBLIES WITH STIFF DIALATORS

(71) Applicant: EDWARDS LIFESCIENCES CORPORATION, Irvine, CA (US)

(72) Inventors: Eitan Atias, Tel Aviv (IL); Noa Axelrod Manela, Kadima (IL); Peleg Harel, Haifa (IL); Ofir Witzman, Harish (IL)

(73) Assignee: EDWARDS LIFESCIENCES CORPORATION, Irvine, CA (US)

(21) Appl. No.: 19/306,125

(22) Filed: Aug. 21, 2025

Related U.S. Application Data
(63) Continuation of application No. PCT/US2024/019500, filed on Mar. 12, 2024.
(60) Provisional application No. 63/451,857, filed on Mar. 13, 2023.

(51) Int. Cl. A61F 2/24; A61M 29/00

(52) U.S. Cl. CPC A61F 2/2433; A61M 29/00

(57) ABSTRACT

The present disclosure relates to tissue perforation assemblies that can be used for perforation and hole dilation through a tissue, such as forming an opening in a host leaflet of a host valvular structure, in which a prosthetic valve can be implanted. In an example, the tissue perforation assembly comprises a delivery nosecone and a dilator distal to the delivery nosecone. Each of the delivery nosecone and the dilator can have a tapering distal portion. A perforating guidewire extends distally from the dilator, and a delivery guidewire extends proximally from the dilator, through a channel of the delivery nosecone. The tissue perforation assembly can be advanced towards the host valvular structure and optionally form a pilot puncture through the host leaflet by the perforating guidewire, after which the dilator can be passed through the puncture to further expand the opening.

### CROSS-REFERENCE TO RELATED APPLICATIONS

This application is a continuation of International Application No. PCT/US2024/019500, filed Mar. 12, 2024, which claims the benefit of U.S. Provisional Application No. 63/451,857, filed Mar. 13, 2023, which is incorporated by reference herein.

### FIELD

The present disclosure relates to tissue perforation assemblies and delivery assemblies for implantation of prosthetic valves, and to methods and devices for modifying existing valvular structures (for example, leaflets of a native heart valve or previously-implanted prosthetic valve) prior to implantation of a guest prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, method that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

In one of its basic configurations, a delivery assembly comprises a delivery apparatus comprising a tissue perforation assembly. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the tissue perforation assembly can comprise a delivery nosecone.

In some examples, the tissue perforation assembly can comprise a dilator.

In some examples, the dilator can comprise a dilator tapering portion between a dilator tapering portion distal end and a dilator tapering portion proximal end.

In some examples, the tissue perforation assembly can comprise a perforating guidewire extending distally from the dilator tapering portion distal end to a guidewire tip.

In some examples, the tissue perforation assembly can comprise a delivery guidewire extending proximally from the dilator tapering portion proximal end.

In some examples, the delivery nosecone can comprise a nosecone channel.

In some examples, the delivery nosecone can comprise a nosecone distal tapering portion between a nosecone distal end and a nosecone tapering portion proximal end.

In one of its basic methods, a method of forming an opening in a target tissue comprises advancing a tissue perforation assembly to a target tissue. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the tissue perforation assembly optionally comprises a dilator distal to a delivery nosecone.

In some examples, the tissue perforation assembly optionally comprises a perforating guidewire attached to the dilator and extending distally therefrom.

In some examples, the tissue perforation assembly optionally comprises a delivery guidewire attached to the dilator and extending proximally therefrom and through a nosecone channel of the delivery nosecone.

In some examples, the method comprises forming, with the perforating guidewire, a pilot puncture within the target tissue.

In some examples, the method comprises advancing the dilator through the pilot puncture to expand the pilot puncture, to form a tissue opening within the target tissue.

In one of its basic configurations, a tissue perforation assembly comprises a dilator, a perforating guidewire, and a delivery guidewire.

In some examples, the tissue perforation assembly can comprise a delivery nosecone.

In some examples, the dilation can optionally be distal to the delivery nosecone.

In some examples, the perforating guidewire can optionally be attached to the dilator.

In some examples, the delivery guidewire can optionally be attached to the dilator.

In some examples, the dilator can comprise a dilator tapering portion extending from a dilator tapering portion distal end and a dilator tapering portion proximal end.

In some examples, the perforating guidewire can optionally extend distally from the dilator tapering portion distal end to a guidewire tip.

In some examples, the delivery guidewire can optionally extend proximally from the dilator tapering portion proximal end.

In some examples, the delivery nosecone can comprise a nosecone channel.

In some examples, the delivery nosecone can comprise a nosecone distal tapering portion extending from a nosecone distal end to a nosecone tapering portion proximal end.

In some examples, the perforating guidewire is optionally configured to pierce through a target tissue so as to form a pilot puncture in the tissue.

In some examples, the dilator is optionally formed from a stiffer material than that of the delivery nosecone.

In some examples, the dilator is optionally configured to expand the pilot puncture when passed through the pilot puncture.

In one of its basic configurations, a delivery assembly comprises a guest prosthetic valve and a delivery apparatus comprising a tissue perforation assembly. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the guest prosthetic valve can comprise a frame movable between a radially compressed and a radially expanded configuration.

In some examples, the delivery apparatus can comprise a handle.

In some examples, the delivery apparatus can comprise a balloon catheter extending from the handle.

In some examples, the delivery apparatus can comprise a balloon mounted on the balloon catheter.

In some examples, the balloon catheter optionally defines a balloon catheter lumen.

In some examples, the balloon is optionally in fluid communication with the balloon catheter lumen.

In some examples, the balloon is optionally configured to transition between deflated and inflated states thereof.

In some examples, the tissue perforation assembly can comprise a delivery nosecone distal to the balloon.

In some examples, the delivery nosecone can comprise a nosecone channel.

In some examples, the delivery nosecone can comprise a nosecone distal tapering portion extending from a nosecone distal end to a nosecone tapering portion proximal end.

In some examples, the tissue perforation assembly can comprise a dilator distal to the delivery nosecone.

In some examples, the tissue perforation assembly can comprise a perforating guidewire.

In some examples, the perforating guidewire is optionally attached to the dilator.

In some examples, the tissue perforation assembly can comprise a delivery guidewire attached to the dilator.

In some examples, the delivery nosecone comprises a nosecone channel.

In some examples, the delivery nosecone comprises a nosecone distal tapering portion extending from a nosecone distal end to a nosecone tapering portion proximal end.

In some examples, the dilator comprises a dilator tapering portion extending from a dilator tapering portion distal end and a dilator tapering portion proximal end.

In some examples, the perforating guidewire extends distally from the dilator tapering portion distal end to a guidewire tip.

In some examples, the delivery guidewire extends proximally from the dilator tapering portion proximal end.

In some examples, the perforating guidewire is optionally configured to pierce through a host leaflet of a host valvular structure so as to form a pilot puncture in the host leaflet.

In some examples, the dilator is optionally configured to pass through the pilot puncture and expand the pilot puncture to form a leaflet opening.

In some examples, when the guest prosthetic valve is disposed around the balloon and positioned within a host valvular structure, inflation of the balloon optionally expands the guest prosthetic valve to implant the guest prosthetic valve in the host valvular structure.

In one of its basic methods, a method of implanting a guest prosthetic valve within a host valvular structure comprises advancing a delivery assembly that comprises a delivery apparatus carrying a guest prosthetic valve in a radially compressed state, to a host valvular structure. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the delivery apparatus can comprise a balloon optionally mounted on a balloon catheter.

In some examples, the delivery apparatus optionally comprises a tissue perforation assembly.

In some examples, the tissue perforation assembly optionally comprises a delivery nosecone distal to the balloon.

In some examples, the tissue perforation assembly optionally comprises a dilator distal to the delivery nosecone.

In some examples, the tissue perforation assembly optionally comprises a perforating guidewire.

In some examples, the perforating guidewire is optionally attached to the dilator.

In some examples, the perforating guidewire optionally extends distally from the dilator.

In some examples, the tissue perforation assembly optionally comprises a delivery guidewire.

In some examples, the delivery guidewire is optionally attached to the dilator.

In some examples, the delivery guidewire optionally extends proximally from the dilator and through a nosecone channel of the delivery nosecone.

In some examples, the method comprises forming, with the perforating guidewire, a pilot puncture within a host leaflet of the host valvular structure.

In some examples, the method comprises advancing the dilator through the pilot puncture to expand the pilot puncture, to form a leaflet opening within the host leaflet.

In some examples, the method optionally comprises positioning the balloon in a deflated state thereof, along with the guest prosthetic valve disposed in a compressed state over the balloon, inside the host valvular structure.

In some examples, the method optionally comprises inflating the balloon so as to radially expand the guest prosthetic valve.

The configurations and methods of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4 shows an exemplary tissue perforation assembly, comprising a perforating guidewire and a delivery guidewire separated from each other.

FIG. 5 shows an exemplary tissue perforation assembly, comprising a perforating guidewire extending through a delivery guidewire.

FIG. 6 shows an exemplary tissue perforation assembly, comprising a delivery guidewire integrally formed with a perforating guidewire.

FIG. 7 shows an exemplary tissue perforation assembly, comprising unitary guidewire having a uniform thickness.

FIG. 8 shows an exemplary tissue perforation assembly, comprising a dilator integrally formed with a perforating guidewire and a delivery guidewire.

FIG. 9A is a simplified side view of a tissue perforation assembly with a perforating guidewire piercing through a host leaflet of a host valvular structure, according to an example.

FIG. 9B is a simplified side view of the tissue perforation assembly of FIG. 9A with a dilator passing through the host leaflet.

FIG. 9C is a simplified side view of the tissue perforation assembly of FIG. 9A with a delivery nosecone passing through the host leaflet.

FIG. 10 shows a delivery assembly comprising a delivery apparatus that carries a prosthetic valve, and includes a tissue perforation assembly.

FIG. 11 is a cross-sectional view of the delivery apparatus of FIG. 10.

FIG. 12A shows the delivery assembly of FIG. 10 passing through a patient's vasculature.

FIG. 12B shows the delivery assembly of FIG. 10 passing through the aortic arch.

FIG. 13A shows the delivery assembly of FIG. 10 with the dilator positioned within a pilot puncture or opening of the host leaflet.

FIG. 13B shows the delivery assembly of FIG. 10 with the delivery nosecone passed through the leaflet opening of the host leaflet.

FIG. 14A is a simplified side view of the delivery assembly of FIG. 10 with a guest prosthetic valve crimped proximal to a deflated balloon which is positioned proximal to the leaflet opening.

FIG. 14B is a simplified side view of the delivery assembly of FIG. 14A with the guest prosthetic valve positioned over the deflated balloon, proximal to the leaflet opening.

FIG. 14C is a simplified side view of the delivery assembly of FIG. 14A with the compressed guest prosthetic valve and the deflated balloon positioned inside the leaflet opening of the host leaflet.

FIG. 14D is a simplified side view of the delivery assembly of FIG. 14A with the guest prosthetic valve expanded by the inflated balloon inside the host valvular structure.

FIG. 15 shows a previously implanted prosthetic valve subsequent to forming the leaflet opening in a host leaflet thereof.

FIG. 16 shows a configuration in which a guest prosthetic valve has been expanded within the leaflet opening of a host prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some examples, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

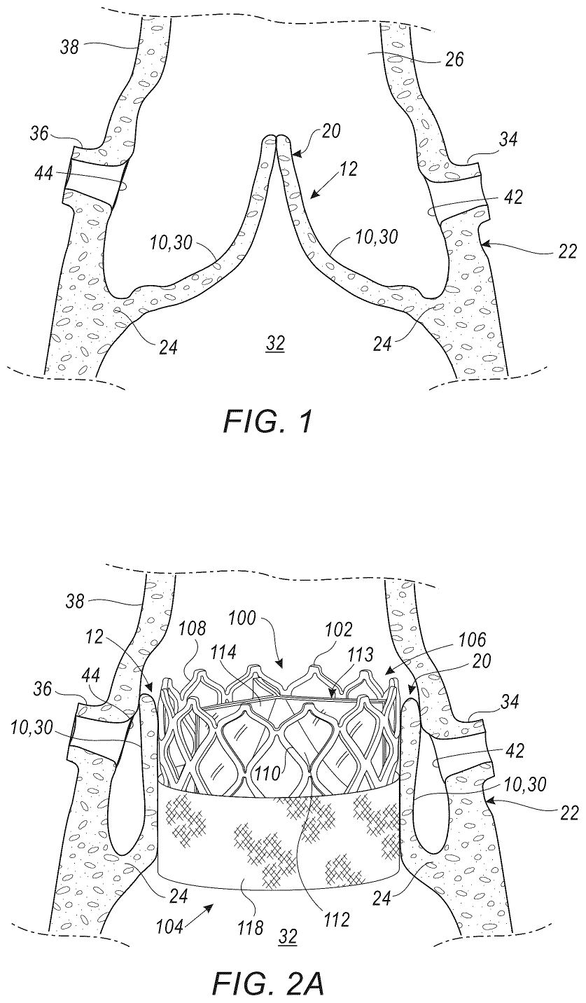

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

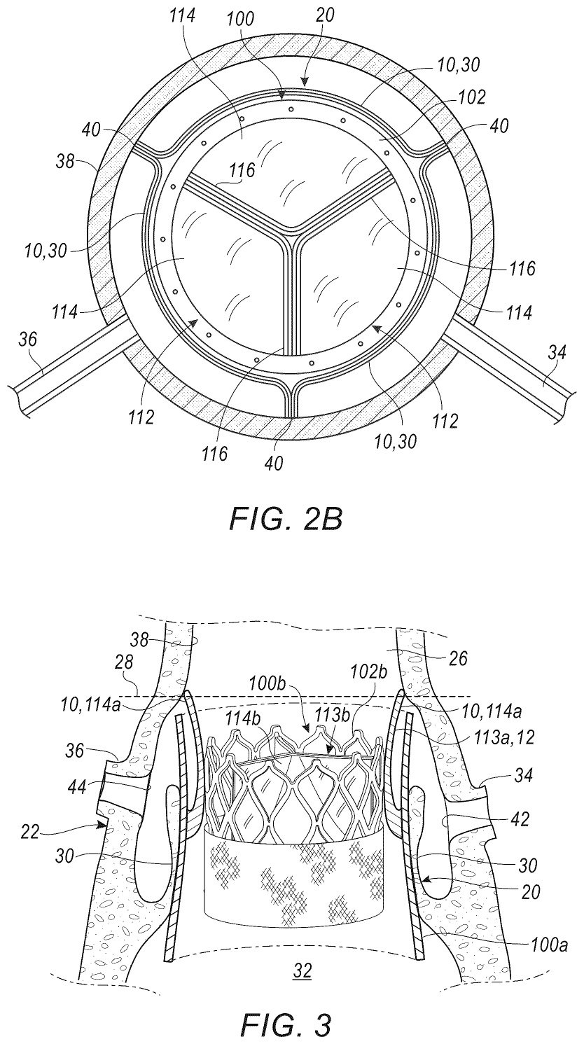

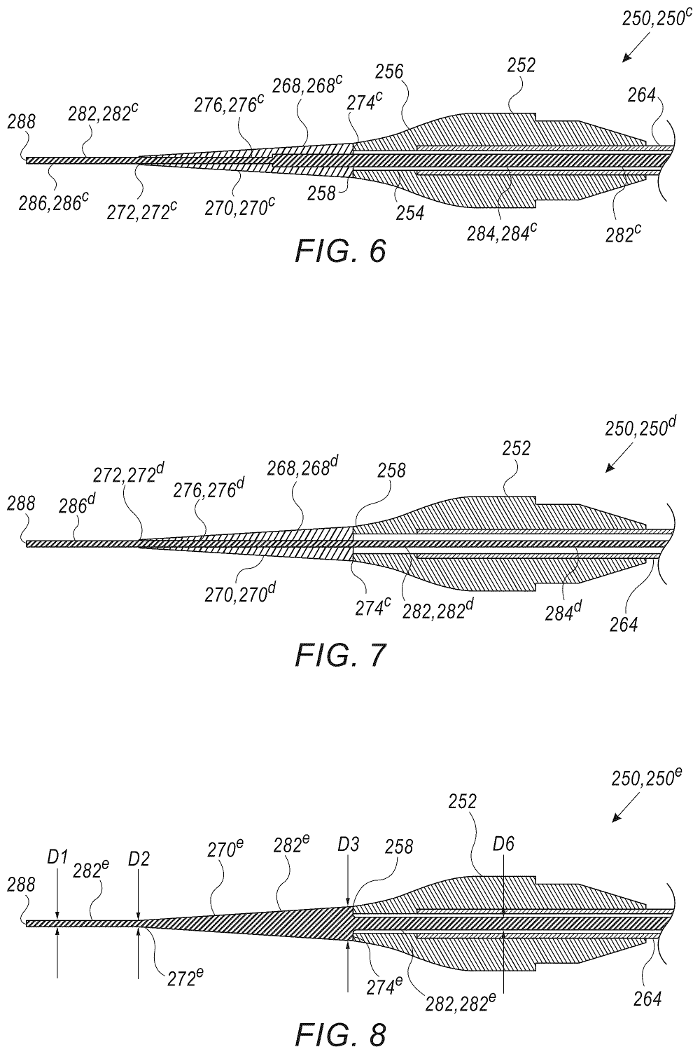

FIGS. 2A-2B show an exemplary prosthetic valve 100 that can optionally be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can optionally be crimped on or retained by an implant delivery apparatus (such as delivery apparatus 202 described below with respect to FIG. 4, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can optionally be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can optionally be made of various suitable materials, including plastically-deformable materials such as, but not limited to, stainless steel, a nickel-based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can optionally be made of shape-memory materials such as, but not limited to, nickel-titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 can optionally be an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can optionally include a plurality of angled struts and vertical or axial struts. At least some of the struts 108 can be pivotable or bendable relative to each other, so as to permit frame expansion or compression. For example, the frame 102 can optionally be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can optionally include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can optionally be arranged together to form prosthetic valve commissures 116 that are coupled (directly or indirectly) to respective portions of the frame 102, thereby securing at least a portion of the valvular structure 113 to the frame 102. The prosthetic valve leaflets 114 can optionally be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can optionally comprise at least one skirt or sealing member. For example, the prosthetic valve 100 can optionally include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can optionally be sutured to the inner skirt along a scalloped line (not shown). An inner skirt can optionally be coupled to the frame 102 via sutures or another form of coupler.

The prosthetic valve 100 can optionally comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can optionally be formed of a single sheet of material that extends continuously around the inner surface of frame 102. In some cases, the outer skirt 118 can optionally be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

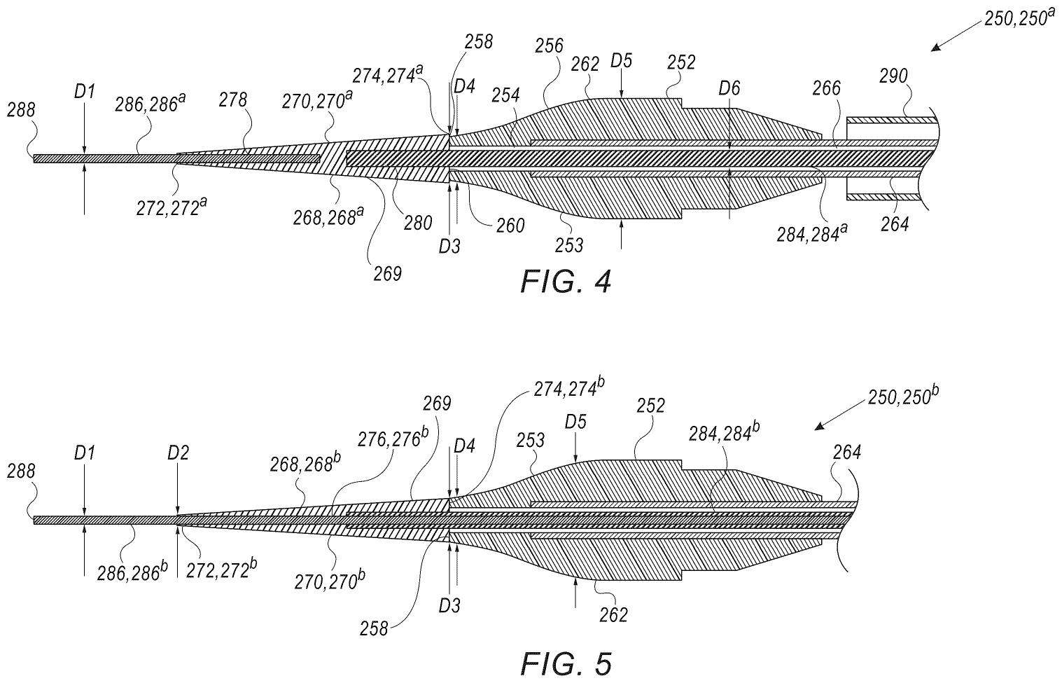

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies cither when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can optionally be a surgically implanted prosthetic heart valve (known as a “surgical valve”) or a transcatheter heart valve. The term “guest valve”, as used herein, refers to a prosthetic valve implanted in a host valve, which can optionally be cither a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted. It is to be understood that any reference to a “prosthetic valve” throughout the disclosure and the claims, without any specific reference to any of the terms “guest” or “host”, refers to a guest prosthetic valve that can be implanted in a native valve or in a previously implanted prosthetic valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and optionally implant a guest prosthetic valve 100 within the modified valvular structure 12, optionally without the need to switch between separate delivery apparatuses for each function.

FIGS. 4-8 show cross-sectional views of examples of a tissue perforation assembly 250 that can optionally be used to perforate and/or cut a tissue, such as a host leaflet 10 of a host valvular structure 12, and to optionally dilate the perforation or cut to expand the opening formed within the tissue. The tissue perforation assembly comprises a delivery nosecone 252, a stiff dilator 268 distal to the delivery nosecone 252, and a perforating guidewire 286 attached to the stiff dilator 268 and terminating at a guidewire tip 288 distal to the stiff dilator 268. The stiff dilator 268 can optionally be conical or frustoconical in shape, and include a dilator tapering portion 270 extending from a dilator tapering portion distal end 272 having a diameter D2 to a dilator tapering portion proximal end 274 having a greater diameter D3, such that D3>D2.

A nosecone shaft 264 defining a nosecone shaft lumen 266 is coupled to the delivery nosecone 252 and extends proximally therefrom. Delivery nosecone 252 can optionally define a nosecone channel 254, which can be optionally continuous with lumen 266 of the nosecone shaft 264, and terminate at a nosecone distal opening 260 at a distal end 258 of the nosecone 252. Delivery nosecone 252 includes a nosecone distal tapering portion 256 which tapers from a diameter D5 of the proximal end 262 to a smaller diameter D4 at the nosecone distal end 258 of the nosecone distal tapering portion 256 (i.e., D5>D4).

The nosecone shaft 264 can optionally be coupled, directly or indirectly, to delivery nosecone 252. In some examples, a distal portion of the nosecone shaft 264 can be optionally coupled to a proximal portion of delivery nosecone 252, as illustrated in FIG. 4. Attachment of the proximal portion of delivery nosecone 252 to a distal portion of nosecone shaft 264 can be achieved by a variety of methods, such as overmolding, radio-frequency welding, through an adhesive, and/or a combination thereof. In some examples (not illustrated), the nosecone shaft 264 can optionally extend through the entire length of the nosecone channel 254, such that a distal end of the nosecone shaft 264 is aligned with the nosecone distal end 258 and defines the nosecone distal opening 260. In some examples (not illustrated), the nosecone shaft 264 is optionally coupled to one or more components, such as collars or other connectors, which are in turn attached to the delivery nosecone 252.

Tissue perforation assembly 250 can optionally further include a delivery guidewire 284 extending through the nosecone shaft lumen 266 and the nosecone channel 254, such that the tissue perforation assembly 250 can optionally be advanced toward the site of treatment in a patient's body, such as a host valvular structure 12, over the delivery guidewire 284. The delivery guidewire 284 can optionally be attached to the stiff dilator 268 and extend proximally therefrom.

The perforating guidewire 286 can optionally be adapted to pierce through a target tissue, so as to form a pilot puncture in the target tissue, such as in a host leaflet 10. In some examples, the guidewire tip 288 can optionally be sharp enough to pierce through a target tissue when forcibly pushed there-against. The perforating guidewire 286 has a diameter D1, which can optionally be uniform at least along the portion extending from the dilator tapering portion distal end 272 to the guidewire tip 288. In some examples, the diameter D2 of the dilator tapering portion distal end 272 is substantially equal to the diameter D1 of the perforating guidewire 286. In some examples, the diameter D2 is not greater than 120% of the diameter D1. In some examples, the diameter D2 is not greater than 110% of the diameter D1. A diameter D2 that approximates diameter D1 can result in a relatively smooth transition between the perforating guidewire 286 and the outer surface 269 of the stiff dilator 268, such that when the perforating guidewire 286 pierces a target tissue, such as a host leaflet 10, further advancement of the tissue perforation assembly 250 into the pilot puncture will allow the tissue surrounding the pilot puncture to climb over the outer surface 269 of stiff dilator 268, such that stiff dilator 268 can expand the opening as it is pushed further therethrough.

Delivery guidewire 284 has a diameter D6, which can optionally be uniform at least along the portion extending proximally from the stiff dilator 268. The nosecone shaft lumen 266, nosecone channel 254 and nosecone distal opening 260 can optionally be sized to allow passage of the delivery guidewire 284 therethrough. In some examples, the delivery guidewire 284 is optionally axially movable relative to the delivery nosecone 252 and/or the nosecone shaft 264. Since the stiff dilator 268 is affixed to the delivery guidewire 284, this means that in such examples, the stiff dilator 268 is similarly axially movable relative to the delivery nosecone 252 and/or the nosecone shaft 264. In some examples, the stiff dilator 268 is optionally attached to the delivery nosecone 252, such as to the nosecone distal end 258, in which case delivery nosecone 252, stiff dilator 268, and delivery guidewire 284 will move in unison when advanced through the patient's vasculature.

In some examples, tissue perforation assembly 250 can optionally further include a catheter 290 through which the nosecone shaft 264 can extend, for example during delivery of the tissue perforation assembly 250 toward the site of treatment. The nosecone shaft 264, as well as delivery nosecone 252, stiff dilator 268 and/or delivery guidewire 284 can optionally be axially movable relative to catheter 290 in such examples. Additional shafts or catheters can optionally be added to tissue perforation assembly 250.

The nosecone distal tapering portion 256 can optionally be relatively flexible, to assist in advancement of the delivery nosecone 252 through the patient's vasculature. Flexibility of the delivery nosecone 252, such as of its distal tapering portion 256, can assist in navigating around a bent or curve in the patient's vasculature. The nosecone 252 might optionally be stiffer at the nosecone tapering portion proximal end 262 or a portion of the nosecone 252 extending proximally from the distal tapering portion 256 due to the increase in thickness at these portions of the nosecone 252. In such cases, increased flexibility of distal tapering portion 256 closer to the nosecone distal end 258 allows a certain length of the distal tapering portion 256 to get around the bend in a patient's vasculature, and create a “follow-the-leader” effect with the remainder of the delivery nosecone 252.

The dilator 268 is optionally formed of a relatively stiff material, which is sufficient to allow passage thereof through a pilot puncture formed in a target tissue, such as a host leaflet 10, without bending due to resistance introduces by the narrow passage of the target tissue is passed through to dilate the opening. In some examples, dilator 268 is optionally stiffer than delivery nosecone 252, or at least from the nosecone distal tapering portion 256. In some examples, dilator 268 is optionally made from a material which is stiffer than that of delivery nosecone 252. Delivery nosecone 252 can optionally be made of a relatively soft polymeric material, such as a material having a hardness in the range from about 50 Shore A to 80 Shore D, and in some examples, in the range from about 60 Shore A to 100 Shore A. Suitable polymeric materials include polyurethane, polyester, polyvinyl chloride, polyethylene, polyamide (nylon), and the like. In contrast, the dilator 268 can optionally be formed of a material having a higher hardness, such as, but not limited to, higher than 80 Shore D. Suitable materials can include polymeric materials and/or metallic materials.

Various exemplary implementations for tissue perforation assemblies 250 can be referred to, throughout the specification, with superscripts, for case of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, tissue perforation assembly 250^{a} is an exemplary implementation of tissue perforation assembly 250, and thus includes all of the features described for tissue perforation assembly 250 throughout the current disclosure, except that while a tissue perforation assembly 250 can include any combination of delivery guidewire 284, perforating guidewire 286, and stiff dilator 268, which can optionally be provided as separate components attached to each other or components integrally formed with each other, the delivery guidewire 284^{a}, perforating guidewire 286^{a}, and stiff dilator 268^{a} of tissue perforation assembly 250^{a} are provided as three separately formed components which are attached to each other, as will be described in further detail below.

FIG. 4 shows cross sectional view of a tissue perforation assembly 250^{a}. As mentioned above, tissue perforation assembly 250^{a} is an exemplary implementation of tissue perforation assembly 250, and thus includes all of the features described for tissue perforation assembly 250 throughout the current disclosure, except that the delivery guidewire 284^{a}, perforating guidewire 286^{a}, and stiff dilator 268^{a} are formed as separate components which are coupled to each other, such that all three can move axially as a single unit after being affixed to each other. In some examples, the stiff dilator 268 can optionally include a distal socket 278 accommodating a proximal end portion of the perforating guidewire 286 therein. In some examples, the stiff dilator 268 can optionally include a proximal socket 280 accommodating a distal end portion of the delivery guidewire 284 therein.

Dilator 268^{a} is shown to include a distal socket 278 extending proximally from dilator tapering portion distal end 272^{a}, and a proximal socket 280 extending distally from dilator tapering portion proximal end 274^{a}, with both sockets 278, 280 axially spaced from each other within dilator 268^{a}. Attachment of the perforating guidewire 286 to the stiff dilator 268, including attachment to distal socket 278, can be achieved by a variety of methods, such as overmolding, radio-frequency welding, through an adhesive, and/or a combination thereof. Similarly, attachment of the delivery guidewire 284 to the stiff dilator 268, including attachment to proximal socket 280, can be achieved by a variety of methods, such as overmolding, radio-frequency welding, through an adhesive, and/or a combination thereof.

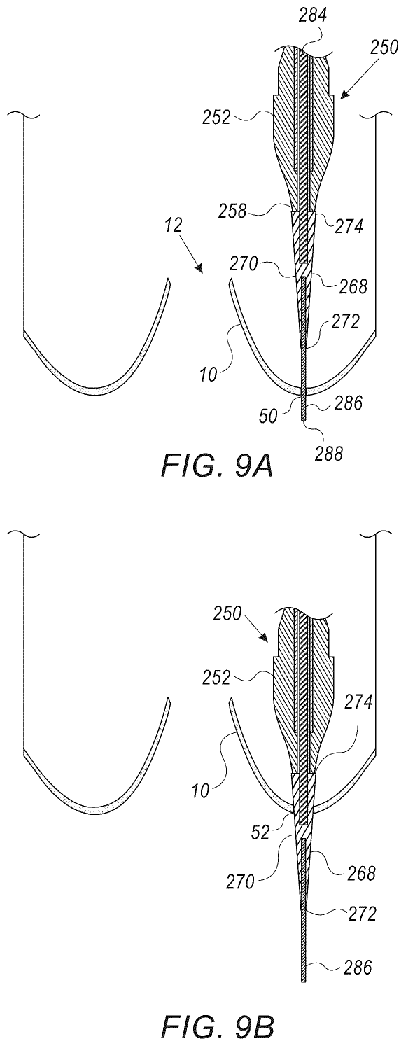

The diameter D3 of dilator tapering portion proximal end 274 can optionally be, in some examples, equal to or greater than the diameter D4 of the nosecone distal end 258. FIG. 5 illustrates an example in which D3=D4, while FIG. 4 shows an example in which D3>D4. It is to be understood that while an exemplary tissue perforation assembly 250^{a} is illustrated in FIG. 4 to include dilator tapering portion proximal end 274 which is somewhat greater in diameter than the nosecone distal end 258, and while an exemplary tissue perforation assembly 250^{b} is illustrated in FIG. 5 to include dilator tapering portion proximal end 274 which is equal in diameter to the nosecone distal end 258, any exemplary tissue perforation assembly 250, including the various examples described herein with respect to FIGS. 4-8, can optionally be implemented such that D3=D4 or such that D3>D4.

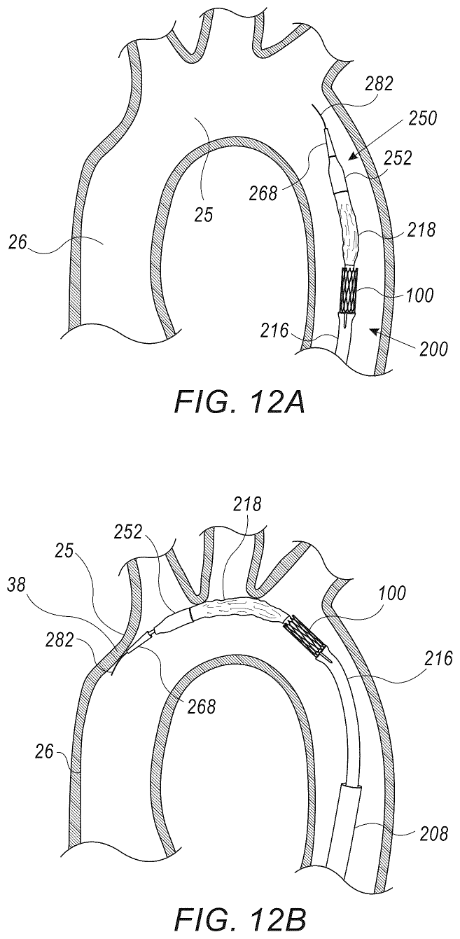

When dilator 268 is passed through a pilot puncture 50 (for example, see FIG. 9A) to form a wider tissue opening 52 (which can be a leaflet opening 52 shown in FIGS. 9B-9C), the nosecone 252 can optionally be kept in contact with dilator 268, for example by keeping the dilator tapering portion proximal end 274 and the nosecone distal end 258 optionally pressed against each other as illustrated throughout FIGS. 4-8, such that as dilator 268 is completely passed past the tissue opening 52, the nosecone 252 can follow and pass through the tissue opening 52 as well, so as to further widen it along the nosecone distal tapering portion 256, up to the diameter D5 of the nosecone tapering portion proximal end 262.

When diameter D3 and D4 are equal to each other, the outer surfaces 269 and 253 of dilator 268 and nosecone 252, respectively, can optionally be continuous and flush with each other, forming together a smooth tapering segment of the tissue perforation assembly 250 along which the tissue around the opening 52 can slide as the tissue perforation assembly 250 is passed therethrough. When diameter D3 is greater than D4, this can optionally form a step-like configuration, such that once dilator 268 is passed past tissue opening 52, the surrounding tissue can slide inward to the smaller diameter D4 of the nosecone distal end 258, and continue to slide therefrom, over nosecone distal tapering portion 256, up to diameter D5 of the nosecone tapering portion proximal end 262.

In some examples, the diameter D4 is not greater than 120% of diameter D3 so as to form a limited step. In some examples, the diameter D4 is not greater than 110% of diameter D3. Diameter D5 is greater than any of diameter D3 and/or D4, even when D3 is slightly greater than D4. In some examples, diameter D3 is at least two times greater than diameter D2. In some examples, diameter D3 is at least two three greater than diameter D2. In some examples, diameter D5 is at least two times greater than diameter D3. In some examples, diameter D5 is at least three times greater than diameter D3.

In some examples, delivery guidewire 284 can optionally have a diameter D6 which is different, and optionally greater than, the diameter D1 of the perforating guidewire 286. This can allow formation of a delivery guidewire 284 with an outer diameter D6 which is similar to that of conventional guidewires, and provides sufficient stiffness to pass the delivery guidewire 284 through the patient vasculature and support advancement of delivery nosecone 252, as well as other components followed thereby, over the delivery guidewire 284. In contrast, the perforating guidewire 286 can optionally be thin enough to facilitate easier penetration thereof through a target tissue, such as a host leaflet 10. In some cases, D6 is greater than D1, for example when forming the entire delivery guidewire 284 with a thin diameter equal to D1 would not allow it to adequately function as a guidewire over which other component of a delivery apparatus or assembly can be advanced, while forming the perforating guidewire 286 with a thick diameter D6 will make it harder to form a pilot puncture in a target tissue. In some examples, D6 is at least 1.5 times greater than D1. In some examples, D6 is at least two times greater than D1.

FIG. 5 shows a cross sectional view of a tissue perforation assembly 250^{b}. Tissue perforation assembly 250^{b} is an exemplary implementation of tissue perforation assembly 250, and thus includes all of the features described for tissue perforation assembly 250 throughout the current disclosure, except that the perforating guidewire 286^{b} is shown to extend through delivery guidewire 284^{b}. Both the perforating guidewire 286^{b} and the delivery guidewire 284^{b} can optionally be attached to the stiff dilator 268^{b}, and extend through a channel 276 formed along the length of stiff dilator 268^{b}. The perforating guidewire 286^{b} can optionally be attached to the delivery guidewire 284^{b}. The delivery guidewire 284^{b} can optionally terminate proximal to dilator tapering portion distal end 2725. The dilator channel 276^{b} can optionally be formed with a stepped configuration, having a wider diameter at a proximal portion thereof, sized to accommodate the delivery guidewire 284^{b}, and a narrower diameter at a distal portion thereof, sized to accommodate the perforating guidewire 286^{b}.

In some examples, the perforating guidewire 286 can optionally include a radio-frequency (RF) energy delivery tip 288 to assist with penetration through the target tissue. For this purpose, a suitable RF energy device may optionally be coupled to the perforating guidewire 286, and the RF energy device can optionally apply the RF energy to the guidewire tip 288 to penetrate the target tissue, such as through a host leaflet 10. In any examples disclosed herein when the perforating guidewire 286 that has a length that extends proximally from dilator 268, constitutes or is attached to a component that can conduct RF energy to the perforating guidewire 286, such as delivery guidewire 284, the perforating guidewire 286 can optionally be coupled, directly or via delivery guidewire 284, to a source of RF energy that applies RF energy to the guidewire tip 288. Thus, delivery guidewire 284^{b} can optionally be coupled, in some examples, to a source of RF energy.

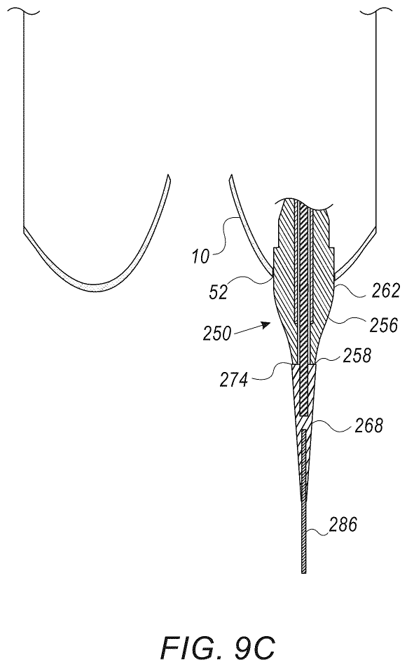

FIG. 6 shows a cross sectional view of a tissue perforation assembly 250^{c}. Tissue perforation assembly 250^{c} is an exemplary implementation of tissue perforation assembly 250, and thus includes all of the features described for tissue perforation assembly 250 throughout the current disclosure, except that the perforating guidewire 286^{c} and delivery guidewire 284^{c} are integrally formed, together forming a unitary guidewire 282. Unitary guidewire 282^{c} can optionally be formed to have two diameters, an outer diameter D1 for a distal portion thereof, serving as the perforating guidewire 286^{c}, and terminating within dilator channel 276^{c}, and a greater outer diameter D6 for a proximal portion thereof, serving as the delivery guidewire 284^{c}, extending proximally from the portion serving as the perforating guidewire 286^{c}. The unitary guidewire 282^{c} is attached to the stiff dilator 268. Dilator channel 276^{c} can optionally be formed with a stepped configuration, similar to that described above with respect to dilator channel 276^{b}. Unitary guidewire 282^{c} can optionally be coupled, in some examples, to a source of RF energy.

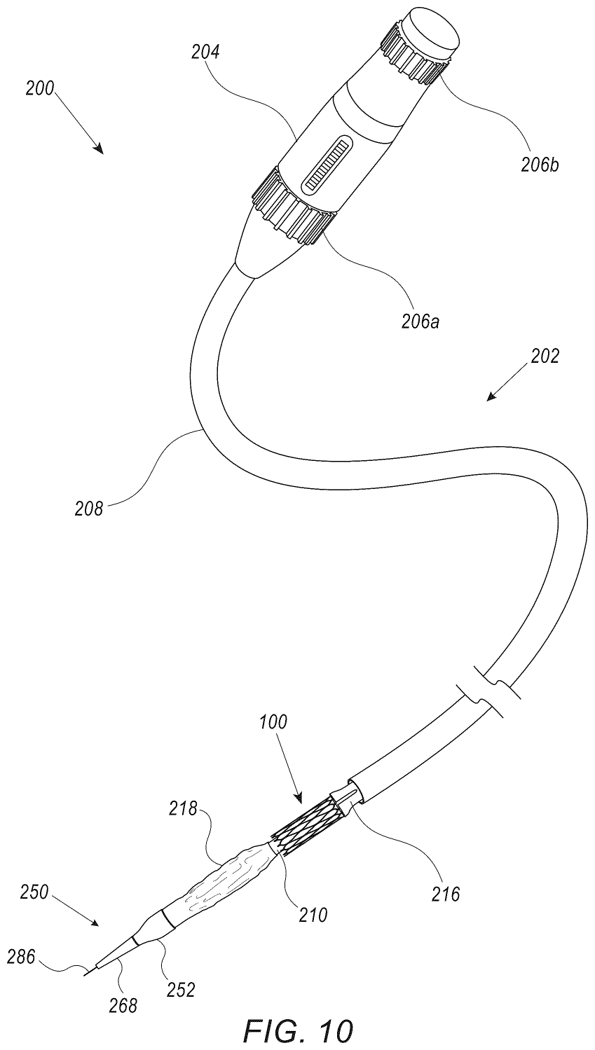

FIG. 7 shows a cross sectional view of a tissue perforation assembly 250^{d}. Tissue perforation assembly 250^{d} is an exemplary implementation of tissue perforation assembly 250, and thus includes all of the features described for tissue perforation assembly 250 throughout the current disclosure. Tissue perforation assembly 250^{d} includes a unitary guidewire 2824, portions of which serve as delivery guidewire 284^{d} and perforating guidewire 2864, in a similar manner to that described above for unitary guidewire 282^{c} with respect to FIG. 6, with the exception that unitary guidewire 282^{d} defines a uniform diameter along its length, which can optionally be similar, for example, to diameter D1 of the perforating guidewire 286 described above with respect to any of the FIGS. 4-6. The perforating guidewire 286^{d} can optionally include or be defined as the portion of the unitary guidewire 282^{d} extending distally from the stiff dilator 268^{d} and terminating at guidewire tip 288. The delivery guidewire 284^{d} can optionally include or be defined as the portion of the unitary guidewire 282^{d} extending proximally from the stiff dilator 268^{d}. Unitary guidewire 282^{d} can optionally be coupled, in some examples, to a source of RF energy.

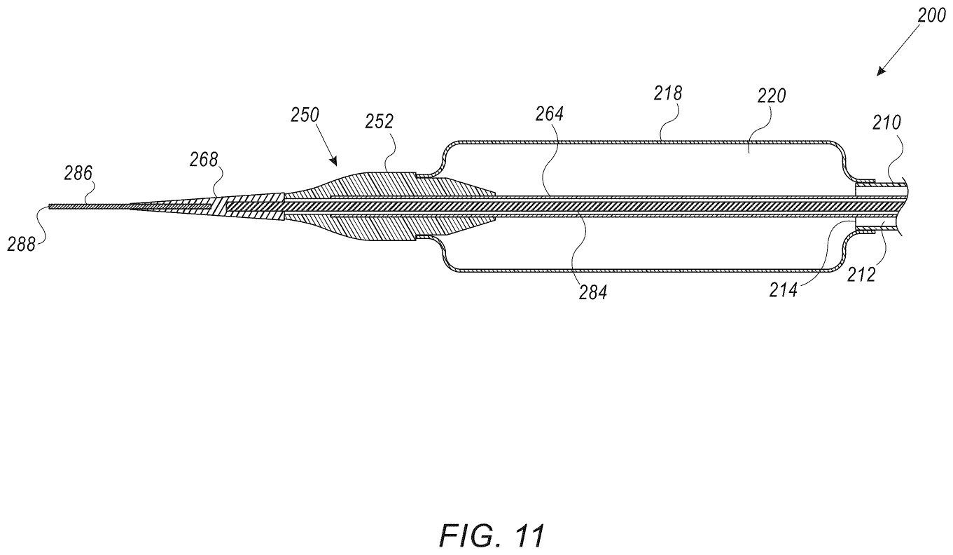

FIG. 8 shows a cross sectional view of a tissue perforation assembly 250ₑ. Tissue perforation assembly 250ₑ is an exemplary implementation of tissue perforation assembly 250, and thus includes all of the features described for tissue perforation assembly 250 throughout the current disclosure, except that the perforating guidewire 286ₑ, dilator 268ₑ and delivery guidewire 284ₑ are integrally formed, together forming a unitary guidewire 282ₑ. Unitary guidewire 282ₑ can optionally be formed to have a non-uniform profile, with varying outer diameters along different portions thereof formed during its manufacturing procedure. A distal portion of the unitary guidewire 282ₑ, having a uniform diameter D1 and terminating at guidewire tip 288, can serve as the perforating guidewire 286ₑ. A tapering portion of the unitary guidewire 282ₑ extending proximally from the perforating guidewire 286ₑ can serve as the stiff dilator 268ₑ, defining a gradual taper that expands from diameter D1 to diameter D3. A proximal portion of the unitary guidewire 282ₑ, having a uniform diameter D6 and extending proximally from stiff dilator 268ₑ, can serve as the delivery guidewire 284ₑ. While the portion forming the delivery guidewire 284ₑ is illustrated in FIG. 8 to have a uniform diameter D6 which is greater than the diameter D1 of the perforating guidewire 286ₑ, it is to be understood that in some examples, the portion forming the delivery guidewire 284ₑ can optionally have a uniform diameter which is substantially equal to the diameter D1 of the perforating guidewire 286ₑ, somewhat similar to the configuration shown in FIG. 7, for example.

FIGS. 9A-9C illustrate some steps in a method for utilizing a tissue perforation assembly 250 for forming an opening within a target tissue. An exemplary implementation of the method is illustrated in FIGS. 9A-9C with respect to forming a leaflet hole inside a host leaflet, which can optionally be performed prior to implanting a guest prosthetic valve inside the host valvular structure. The tissue perforation assembly 250 can optionally be used to perforate, cut, and/or tear a host leaflet 10, such as a native leaflet 30 or a prosthetic valve leaflet 114 of a previously implanted prosthetic valve. While tissue perforation assembly 250^{a} is illustrated throughout FIGS. 9A-9C, it is to be understood that other examples of tissue perforation assemblies 250 described in the current specification can optionally be used in a similar manner.

The distal end portion of the tissue perforation assembly 250, is configured to be inserted into a patient's vasculature, such as within an ascending aorta, and to be advanced towards the host leaflet 10. Positioning tissue perforation assembly 250 relative to the host leaflet 10 may optionally comprise advancing the tissue perforation assembly 250 toward the leaflet via delivery guidewire 284. As mentioned above, the perforating guidewire 286 is configured to pierce a target tissue, such as host leaflet 10 of a host valvular structure 12, to form a pilot puncture 50 in the host leaflet 10, as shown in FIG. 9A.

The delivery guidewire 284 can optionally be coupled to a handle. The handle can optionally have one or more actuators that are operatively coupled to the delivery guidewire 284 to facilitate axial movement thereof. Since delivery guidewire 284 is attached to dilator 268, which is attached to perforating guidewire 286, either by being interconnected or by being integrally formed, axial movement of delivery guidewire 284 serves to move the dilator 268 and perforating guidewire 286 therewith. In some examples, the guidewire tip 288 can optionally be configured to pierce a host leaflet 10 when axially translated so as to contact the host leaflet, at which point the guidewire tip 288 can optionally be forcibly pushed to as to puncture through the host leaflet 10, and/or direct RF energy to form a puncture within the host leaflet 10.

FIG. 9A illustrates an example in which the perforating guidewire 286 punctures or lacerates the host leaflet 10 to form a pilot puncture 50 within host leaflet 10. Subsequent to forming the pilot puncture 50, and as shown in FIG. 9B, the stiff dilator 268 can optionally be inserted into the pilot puncture 50 to expand the pilot puncture 50 to form a leaflet opening 52. As the dilator 268 is inserted into the host leaflet 10, the inherent resiliency of the leaflet 10 may urge the leaflet 10 radially inwardly against the dilator 268. The increased stiffness of dilator 268 facilitates advancement thereof through the leaflet 10, wherein the gradually tapering shape of the dilator 268 facilitates expanding the leaflet opening 52 to a greater diameter.

The delivery nosecone 252 can optionally be kept in contact with dilator 268 during advancement through the host leaflet 10, such that as dilator 268 is completely passed through the leaflet opening 52, the delivery nosecone 252 is inserted into the leaflet opening 52, as shown in FIG. 9C, and can optionally be passed in a continuous manner therethrough, wherein the gradually tapering shape of the nosecone distal tapering portion 256 facilitates further expansion of the leaflet opening 52 to a diameter which is greater than that formed by the dilator 268.

In some examples, the delivery nosecone 252 can optionally be axially spaced from the dilator 268, in which case the leaflet opening 52 formed by dilator 268 can optionally be sufficiently large to allow insertion of the nosecone distal end 258 therethrough, and advancement of the nosecone 252 as described above to facilitate further expansion of the leaflet opening to a greater diameter.

In some examples, passing components of the tissue perforation assembly 250 such as dilator 268 and/or nosecone 252 through the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

The tissue perforation assembly 250 may be configured to form the leaflet opening 52 in any of a variety of host valvular structures 12. In the example of FIGS. 9A-9C, the host valvular structure 12 can optionally be the valvular structure 113 of a previously implanted prosthetic valve, such as the prosthetic valve 100a of FIG. 3. In such examples, using the delivery assembly 200 as described herein to form the leaflet opening 52 in a previously implanted prosthetic valve may be optionally followed by steps for implanting a guest prosthetic valve 100b within the previously implanted prosthetic valve 100a (for example, via a ViV procedure).

Similarly, the host valvular structure 12 in the example of FIGS. 9A-9C can optionally be a valvular structure 29 of a native heart valve, such as the native aortic valve 20 shown in FIGS. 2A-2B. In such examples, the tissue perforation assembly 250 can be optionally configured to puncture a native leaflet 30 of the native aortic valve 20. In some examples, the host valvular structure and/or the native valve may refer to another valve of a patient's heart, such as a mitral valve, a pulmonary valve, or a tricuspid valve.

While illustrated and described above with respect to forming a leaflet opening 52 within a host leaflet 10, it is to be understood that the tissue perforation assembly 250 may optionally be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some examples, a tissue perforation assembly 250 may optionally be utilized to form an opening through the interatrial septum, for example at the site of the fossa ovalis, which is a region of the septum containing tissue of lesser thickness than is typical of the rest of the septum. Thus, any example of tissue perforation assembly 250 described above can optionally be utilized in a manner similar to that described with respect to FIGS. 9A-9C, to form a tissue opening, equivalent to leaflet opening 52 described with respect to FIG. 9A-9C, in a target tissue, equivalent to a host leaflet 10 described with respect to FIGS. 9A-9C.

In some examples, tissue perforation assembly 250 can optionally be part of a delivery assembly that includes a delivery apparatus carrying a prosthetic valve. Any delivery assembly disclosed herein, comprises a delivery apparatus that can include a tissue perforation assembly according to any of the examples described above, and a balloon expandable prosthetic valve. While examples of a delivery assembly described in the current disclosure, are shown to include an exemplary delivery apparatus and a balloon expandable prosthetic valve, it should be understood that a delivery apparatus that includes a tissue perforation assembly according to any example of the current disclosure can optionally be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

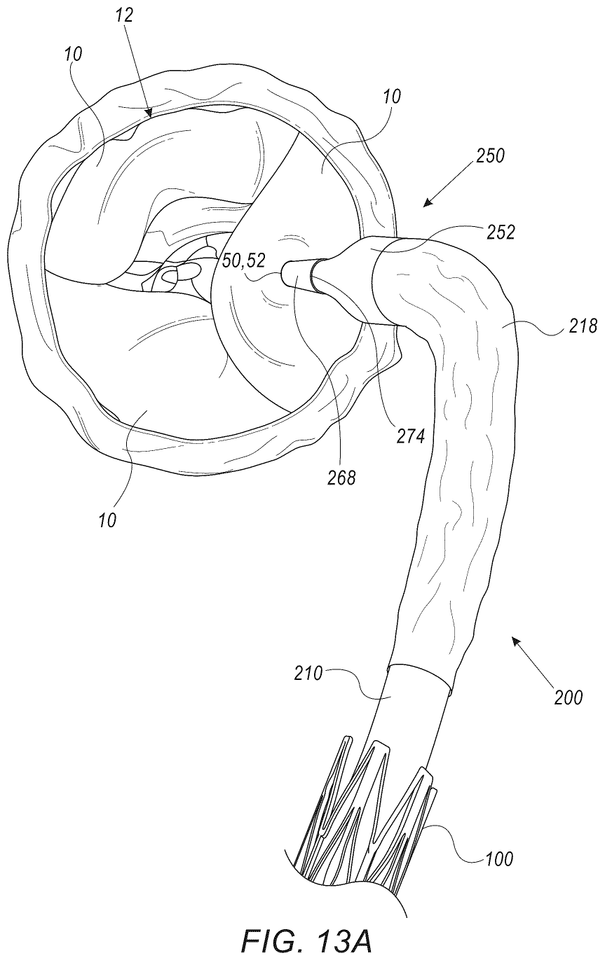

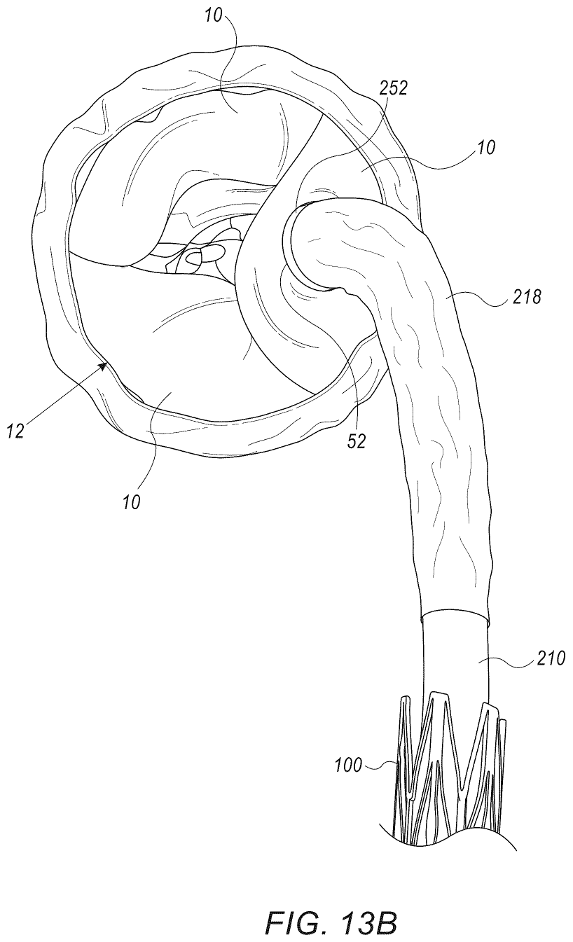

FIG. 10 illustrates an exemplary delivery assembly 200 that includes an exemplary delivery apparatus 202 adapted to deliver a balloon expandable prosthetic valve 100, such as prosthetic valve 100 described above with respect to FIGS. 2A-2B. FIG. 11 shows a cross-sectional view of a distal portion of delivery apparatus 202 comprising a tissue perforation assembly 250. It is to be understood that a tissue perforation assembly 250^{a} is shown in FIG. 11 by way of illustration and not limitation, and that the delivery apparatus 202 includes a tissue perforation assembly 250 which can optionally be implemented according to any of the examples described above. According to some examples, the delivery apparatus 202 further includes a handle 204 and a balloon catheter 210 having a balloon 218 mounted on its distal end, proximal to the delivery nosecone 252. The balloon expandable prosthetic valve 100 can optionally be carried in a crimped state over the balloon catheter 210 or over another catheter disposed around the balloon catheter 210. Optionally, an outer delivery shaft 208 can concentrically extend over the balloon catheter 210, and a push shaft 216 can be disposed over the balloon catheter 210, optionally between the balloon catheter 210 and the delivery shaft 208. Any of the balloon catheter 210 or the outer delivery shaft 208 can optionally be the catheter 290 described above with respect to tissue perforation assemblies 250. The nosecone shaft 264 can optionally extend through the lumen 212 of the balloon catheter 210 and an internal cavity 220 of the balloon 218 towards the handle 204.

The outer delivery shaft 208, the push shaft 228, the balloon catheter 210, and the nosecone shaft 264, can optionally be configured to be axially movable relative to each other. For example, a proximally oriented movement of the outer delivery shaft 208 relative to the balloon catheter 210, or a distally oriented movement of the balloon catheter 210 relative to the outer delivery shaft 208, can optionally expose the prosthetic valve 100 from the outer delivery shaft 208.

The proximal ends of the balloon catheter 210, the outer delivery shaft 208, the push shaft 216, and the nosecone shaft 264, can optionally be coupled to the handle 204. During delivery of the prosthetic valve 100, the handle 204 can optionally be maneuvered by an operator (for example, a clinician or a surgeon) to axially advance or retract components of the delivery apparatus 202, such as the delivery guidewire 284, the nosecone shaft 264, the balloon catheter 210, the outer delivery shaft 208, and/or the push shaft 216, through the patient's vasculature and/or along the target site of implantation, as well as to inflate the balloon 218 mounted on the balloon catheter 210 so as to expand the prosthetic valve 100, and to deflate the balloon 218 and retract the delivery apparatus 202 once the guest prosthetic valve 100 is mounted in the implantation site (for example, within the host valve).

The balloon catheter 210 can extend through the handle 204 and optionally be fluidly connectable to a fluid source for inflating the balloon 218. The fluid source comprises an inflation fluid. The term “inflation fluid”, as used herein, means a fluid (for example, saline, though other liquids or gas can be used) used for inflating the balloon 218. An inflation fluid source is in fluid communication with the balloon catheter lumen 212, such as the annular space between the inner surface of balloon catheter 210 and the outer surface of nosecone shaft 264 extending therethrough, such that fluid from the fluid source can optionally flow through the balloon catheter lumen 212, and into the balloon 218 to inflate it.

The handle 204 can optionally include a steering mechanism configured to adjust the curvature of the distal end portion of the delivery apparatus 202. In the illustrated example, the handle 204 can optionally include an adjustment member, such as the illustrated rotatable knob 206a, which in turn is operatively coupled to the proximal end portion of a pull wire. The pull wire can optionally extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can optionally further include an adjustment mechanism including an adjustment member, such as the illustrated rotatable knob 206b. The adjustment mechanism can be configured to adjust the axial position of the push shaft 216 relative to the outer balloon catheter. The handle can optionally include additional adjustment mechanisms controllable by additional knobs to maneuver additional components of the delivery apparatus 202, such as axial movement of the delivery guidewire 284, axial movement of the nosecone shaft 264, axial movement of the balloon catheter 210, and/or axial movement of a push shaft 216, relative to other shafts, wires or components of the delivery apparatus 202.

The prosthetic valve 100 can optionally be carried by the delivery apparatus 202 during delivery in a crimped state, and expanded by inflation of balloon 218 to secure it in a native heart valve annulus (such as aortic annulus 24) or against a previously implanted prosthetic valve. In an exemplary implantation procedure, the prosthetic valve 100 can optionally be initially crimped over the balloon catheter 210, proximal to the balloon 218. Because prosthetic valve 100 is crimped at a location different from the location of balloon 218, prosthetic valve 100 can be crimped to a lower profile than would be possible if it was crimped on top of balloon 218. This lower profile permits the clinician to more easily navigate the delivery assembly 200 (including crimped prosthetic valve 100) through a patient's vasculature to the treatment location. The lower profile of the crimped prosthetic valve is particularly helpful when navigating through portions of the patient's vasculature which are particularly narrow, such as the iliac artery.

When reaching the host valve, the delivery apparatus 202 can be utilized to modify at least one host leaflet 10 described above with respect to FIGS. 9A-9C, after which the deflated balloon 218, carrying crimped prosthetic valve 100 thereover, can be advanced to the host valvular structure 12 to expand the guest prosthetic valve 100. Prior to inflation of balloon 218, the push shaft 216 can optionally be advanced distally, allowing its distal end portion to contact and push against the outflow end 106 of prosthetic valve 100, pushing the prosthetic valve 100 distally therewith. The distal end of push shaft 216 is dimensioned to engage with the outflow end 106 of prosthetic valve 100 in a crimped configuration of the valve. In some examples, the distal end portion of the push shaft 216 can optionally be flared radially outward, to terminate at a wider-diameter that can contact the prosthetic valve 100 in its crimped state. Optionally, push shaft 216 can then be advanced distally, pushing the prosthetic valve 100 therewith, until the crimped prosthetic valve 100 is disposed around the balloon 218, after which the balloon 218 can optionally be inflated to radially expand the prosthetic valve 100. Once the prosthetic valve 100 is expanded to its functional diameter within a native annulus or within a previously implanted host prosthetic valve, the balloon 218 can optionally be deflated and the delivery apparatus 202 can optionally be retrieved from the patient's body.

In some examples, the delivery assembly 200 can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can optionally be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Nosecone shaft 264, balloon catheter 210, and optional outer delivery shaft 208, can optionally be formed from any of various suitable materials, such as nylon, braided stainless steel wires, or a polyether block amide (commercially available as Pebax®). In some examples, nosecone shaft 264, balloon catheter 210, and optional outer delivery shaft 208, can optionally have longitudinal sections formed from different materials in order to vary the flexibility of the shafts along their lengths. In some examples, nosecone shaft 264 has an inner liner or layer formed of Teflon® to minimize sliding friction with delivery guidewire 284.

The nosecone shaft 264 can optionally be sized such that an annular space is formed within balloon catheter lumen 212 between balloon catheter 210 and nosecone shaft 264 along the length of balloon catheter 210. This annular space is optionally in fluid communication with one or more balloon catheter openings 214 exposed to an internal cavity 220 of the balloon 218, which can optionally be in fluid communication with a fluid source (for example, a syringe or a pump) that can optionally inject an inflation fluid (for example, saline) into balloon cavity 220. In this way, fluid from the fluid source can optionally flow through balloon catheter lumen 212, and into balloon cavity 220 via balloon catheter opening(s) 214, which serves to inflate the balloon 218 and expand and deploy a prosthetic valve 100 disposed thereon. The pressure of the inflation fluid within balloon cavity 220 may provide the force that allows the balloon 218 to expand a prosthetic valve 100 disposed thereon. Further, the balloon catheter lumen 212 may optionally be configured to withdraw fluid from balloon cavity 220 through balloon catheter opening(s) 214, to deflate the balloon 218.

While balloon catheter 210 is shown to terminate at a proximal end of balloon 218 in FIG. 11, in some examples, balloon catheter 210 can optionally extend farther in the distal direction (examples not illustrated), through a portion or through the entire length of balloon cavity 220, and one or more balloon catheter opening(s) 214 can optionally be formed on the sidewall of balloon catheter 210, exposed laterally to balloon cavity 220.

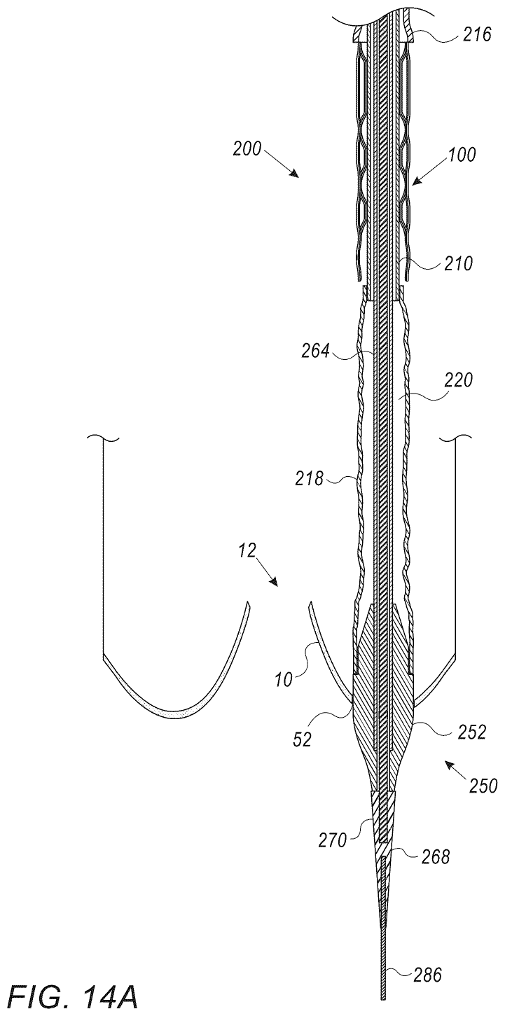

FIGS. 12A-14D show stages in an exemplary implantation procedure for implanting the prosthetic valve 100 within a host leaflet structure 12 using a transfemoral delivery procedure. In some examples, various other delivery procedures can optionally be used, such as transventricular, transapical, transseptal, etc. Delivery apparatus 202 may include any of a variety of features to facilitate positioning the perforating guidewire 286, stiff dilator 268, and/or the delivery nosecone 252, relative to the host leaflet 10. For example, the nosecone shaft 264, balloon catheter 210, outer delivery shaft 208, delivery guidewire 284, and/or the perforating guidewire 286, may optionally be pre-formed, shaped, and/or curved so as to be directed and/or angled toward the host leaflet 10 when positioned in the vicinity of the host valvular structure 12. Furthermore, one or more shafts of delivery apparatus 202, such as outer delivery shaft 208, can optionally have a steering mechanism (for example, a pull wire and a corresponding adjustment mechanism in the handle 204) to steer or adjust its distal end.

Referring to FIG. 12A, the distal end portion of the delivery assembly 200 is inserted into the vasculature of a patient such that balloon catheter 210, delivery nosecone 252 and dilator 268 extend through the patient's aorta 26 and approach the aortic arch 25. As shown in FIG. 12B, as an axial force is applied to the delivery apparatus 202 (for example, at the handle 204) to advance the delivery apparatus 202 around the aortic arch 25, the dilator 268 and delivery nosecone 252 may optionally contact the surrounding aortic wall 38. Collectively, the dilator 268 and delivery nosecone 252 can optionally form a rigid section if together formed as a unitary component formed of the same material and/or having a similar stiffness.

Thus, if both the dilator 268 and the delivery nosecone 252 would have been formed as portions of a single unit, such a unit would form a rigid section of the delivery assembly which lacks any significant flexibility and therefore, navigating the aortic arch 25 would force such a unitary rigid elongated structure to twist and/or rotate relative to the nosecone shaft as it contacts the aortic wall 38. The rigidity of such an elongated unitary rigid section would therefore require a greater axial force to circumvent the aortic arch 25, thereby causing high frictional forces against the aortic wall 38, potentially damaging native tissue. However, the tissue perforation assembly 250 disclosed herein, includes a separate dilator 268 which is stiffer than delivery nosecone 252, thus adding bendability to the section of the delivery apparatus 202 that includes both cones 268, 252, limiting the friction generally created to navigate the delivery apparatus 202 around the aortic arch 25.

Referring to FIG. 12B, the stiff nosecone 268 has low bendability and/or flexibility and thus remains in a relatively linear unbent configuration even when pressed against the aortic wall. However, the separate delivery nosecone 252 has increased bending flexibility relative to the stiff dilator 268, allowing at least a portion of nosecone 252, such as its distal tapering portion 256, to bend when forcibly pressed against the aortic wall. Thus, the overall length of a stiff portion of the tissue perforation assembly 250 is limited to the length of the stiff dilator 268, which is significantly less than the combined length of dilator 268 and nosecone 252.

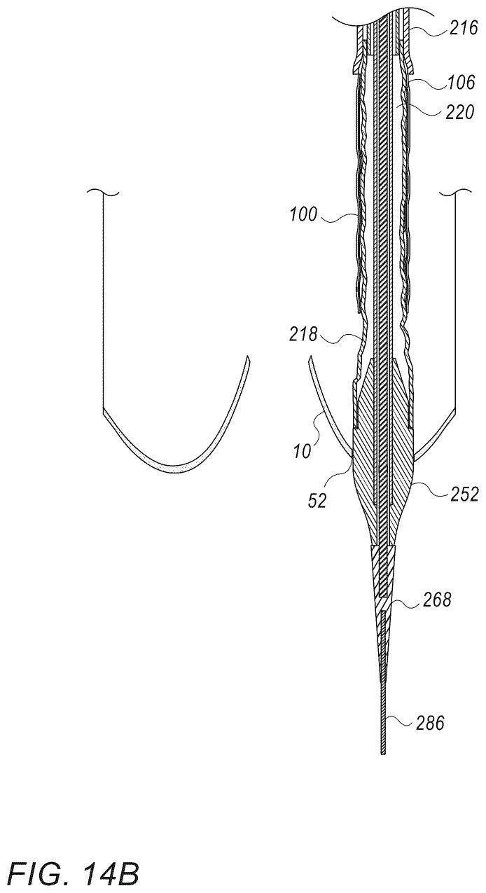

Formation of a leaflet opening 52 in a host leaflet 10 by a tissue perforation assembly 250 of a delivery assembly 200 can be performed in the same manner described above with respect to FIGS. 9A-9C. FIGS. 13A-13B illustrate the tissue perforation assembly 250 utilized to expand the pilot puncture 50 into the leaflet opening 52. In particular, FIG. 13A illustrates the stiff dilator 268 positioned within and passing through the pilot puncture 50, so as to enlarge it into leaflet opening 52, corresponding to the state described above with respect to FIG. 9B, while FIG. 13B illustrates the delivery nosecone 252 passed through the leaflet opening 52 formed by the dilator 268 so as to further enlarge the leaflet opening 52, corresponding to the state described above with respect to FIG. 9C. As shown throughout FIGS. 13A-13B, the balloon 218, positioned proximal to the nosecone 252, remains in a deflated state throughout steps of the procedure equivalent to those described above with respect to FIGS. 9A-9C.

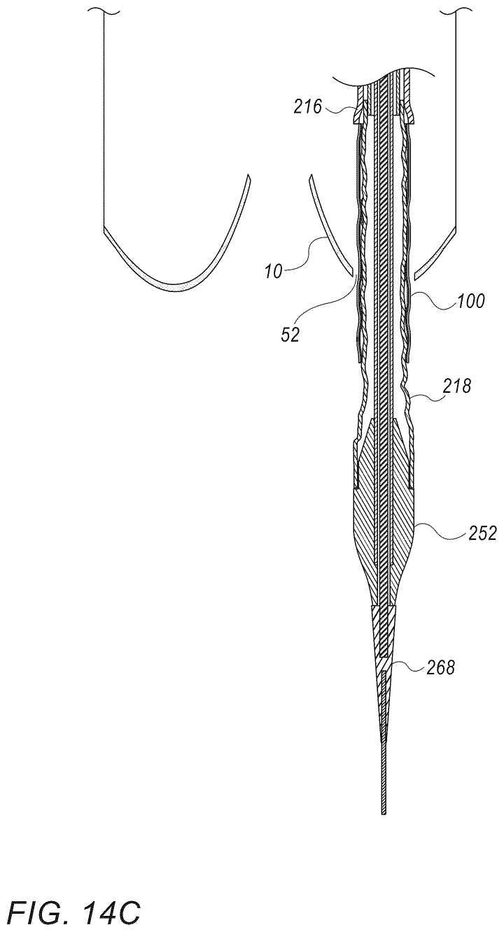

FIGS. 14A-14D show subsequent steps of a method utilizing delivery assembly 200, following steps equivalent to those described above and illustrated in FIGS. 9A-9C. After formation of the leaflet opening 52 by stiff dilator 268, and optional further enlargement of the opening 52 by the nosecone 252 as shown in FIGS. 9C and 14A, the push shaft 216 can optionally be utilized to distally advance the crimped prosthetic valve 100 toward and around balloon 218, as shown in FIG. 14B.

The deflated balloon 218 and the prosthetic valve 100 disposed thereover can be then optionally advanced and positioned within the leaflet opening 52, as shown in FIG. 14C. The push shaft 216 can optionally remain in position, abutting outflow end 106 of prosthetic valve 100 during advancement into and through the leaflet opening 52, to provide a counter force that resists proximal displacement of the prosthetic valve 100 during insertion into leaflet opening 52.

At this stage, delivering inflation fluid into the balloon cavity 220 allows the balloon 218 to inflate and expand the prosthetic valve 100, as shown in FIG. 14D. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 14B to be pushed distally to the position of balloon 218 after forming the leaflet opening 52, it is to be understood that the prosthetic valve 100 can optionally be pushed by push shaft 216 over balloon 218 at any other stage prior to advancement of the balloon 218 into the leaflet opening 52 as shown in FIG. 14C. For example, the prosthetic valve 100 can optionally be pushed over balloon 218 upon approximation to the site of implantation, such as upon reaching the region of the ascending aorta 26 even prior to forming the pilot puncture as shown in FIG. 9A. Similarly, the prosthetic valve 100 can optionally be pushed distally after the pilot puncture 50 is formed but before advancing the stiff dilator 268 into the pilot puncture 50, or after positioning the stiff dilator 268 inside the pilot puncture 50 but before passing it completely through the leaflet 10 to form the leaflet opening 52, or after forming the leaflet opening 52 but before positioning the delivery nosecone 252 in leaflet opening 52, or after passing at least a portion of the nosecone 252 through the leaflet 10 to enlarge leaflet opening 52. Similarly, it is to be understood that in some examples, the delivery assembly can optionally be provided without a push shaft 216, and/or that the prosthetic valve 100 can optionally be crimped around the outer balloon 218 and delivered through the patient's vasculature in this position.

With the guest prosthetic valve 100 received within the leaflet opening 52, radially expanding the guest prosthetic valve, as shown in FIG. 14D, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

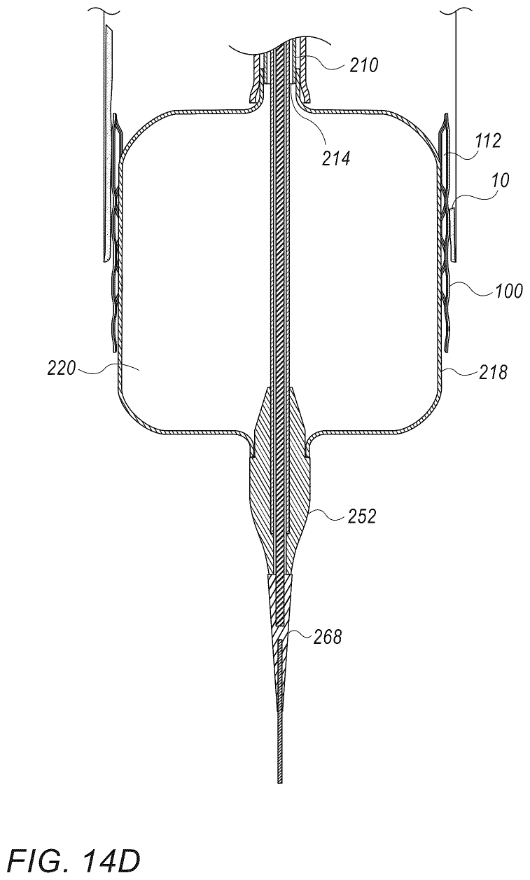

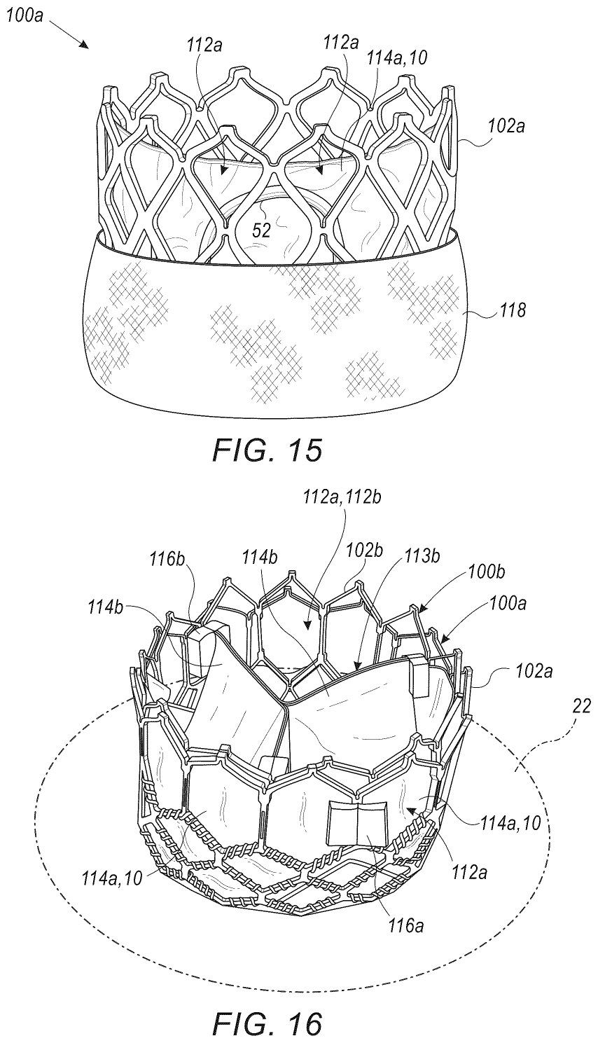

As mentioned, the delivery assemblies and methods of the current specification can optionally be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 15 shows a previously implanted prosthetic valve 100a subsequent to forming the leaflet opening 52, for example subsequent to the method described above with respect to FIGS. 9A-9C. FIG. 16 shows a configuration in which a second prosthetic valve 100b has been expanded within the leaflet opening 52 of a host prosthetic valve 100a. In the example of FIG. 16, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 15, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 16, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can optionally comprise, in some examples, repeating one or more steps disclosed throughout the current specification to form a plurality of punctures and openings in the host valvular structure. For example, steps described above with respect to FIGS. 9A-9C can be performed for forming a first leaflet opening in a first host leaflet, after which the delivery apparatus, including perforating guidewire 286, stiff dilator 268, and/or delivery nosecone 252, can optionally be retracted from the first host leaflet and steered toward another host leaflet, after which the same or equivalent steps can optionally be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In some examples, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can optionally be modified such that the guest prosthetic valve can optionally be implanted by being expanded in a leaflet opening of a first host leaflet that faces the left coronary artery, and such that the second leaflet opening can optionally be formed in a second host leaflet that faces the right coronary artery (or vice-versa).

In some examples, forming the first leaflet opening can optionally be performed prior to forming the second leaflet opening. In some examples, forming the second leaflet opening can optionally be performed prior to forming the first leaflet opening. In some examples, the order of forming leaflet openings is chosen such that the final leaflet opening is formed in the host leaflet in which the guest prosthetic valve 100 is to be positioned and expanded, such as over a balloon as described above with respect to FIGS. 14A-14D.

It is to be understood that the guest prosthetic valve 100 is not limited to being implanted within an opening 52 of a leaflet. For example, in cases where the tissue perforation assembly 250 forms a full tear in a host leaflet that extends to the coaptation edge of the leaflet, the guest prosthetic valve 100 can optionally be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic heart valve can be positioned at a location between the leaflets of the host valvular structure 12 (such that the delivery assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A delivery assembly comprising: a delivery apparatus comprising a tissue perforation assembly, the tissue perforation assembly comprising: a delivery nosecone comprising: a nosecone channel; and a nosecone distal tapering portion between a nosecone distal end and a nosecone tapering portion proximal end; a dilator comprising a dilator tapering portion between a dilator tapering portion distal end and a dilator tapering portion proximal end; a perforating guidewire extending distally from the dilator tapering portion distal end to a guidewire tip; and a delivery guidewire extending proximally from the dilator tapering portion proximal end.

Example 2. The delivery assembly of any example herein, particularly example 1, wherein the dilator is distal to the delivery nosecone.

Example 3. The delivery assembly of any example herein, particularly example 1 or 2, wherein the perforating guidewire is attached to the dilator.

Example 4. The delivery assembly of any example herein, particularly any one of examples 1 to 3, wherein the delivery guidewire is attached to the dilator.

Example 5. The delivery assembly of any example herein, particularly any one of examples 1 to 4, wherein the dilator is formed from a stiffer material than that of the delivery nosecone.

Example 6. The delivery assembly of any example herein, particularly any one of examples 1 to 4, wherein the tissue perforation assembly further comprises a nosecone shaft attached to the delivery nosecone and extending proximally therefrom, the nosecone shaft defining a nosecone shaft lumen sized to allow axial passage of the delivery guidewire therethrough.

Example 7. The delivery assembly of any example herein, particularly any one of examples 1 to 6, wherein a diameter defined by the dilator tapering portion distal end is not greater than 120% of a diameter defined by the perforating guidewire.

Example 8. The delivery assembly of any example herein, particularly example 7, wherein the diameter of the dilator tapering portion distal end is not greater than 110% of the diameter of the perforating guidewire.

Example 9. The delivery assembly of any example herein, particularly any one of examples 1 to 8, wherein a diameter defined by the dilator tapering portion proximal end is equal to or greater than a diameter defined by the nosecone distal end.

Example 10. The delivery assembly of any example herein, particularly example 9, wherein the diameter of the dilator tapering portion proximal end is not greater than 120% of the diameter of the nosecone distal end.

Example 11. The delivery assembly of any example herein, particularly example 9, wherein the diameter of the dilator tapering portion proximal end is not greater than 110% of the diameter of the nosecone distal end.

Example 12. The delivery assembly of any example herein, particularly any one of examples 9 to 11, wherein a diameter defined by the nosecone tapering portion proximal end is greater than the diameter of the dilator tapering portion proximal end.

Example 13. The tissue perforation assembly of any example herein, particularly any one of examples 1 to 12, wherein the perforating guidewire and the delivery guidewire are axially spaced from each other.

Example 14. The delivery assembly of any example herein, particularly example 13, wherein the dilator comprises a distal socket to which the perforating guidewire is attached.

Example 15. The delivery assembly of any example herein, particularly example 13 or 14, wherein the dilator comprises a proximal socket to which the delivery guidewire is attached.

Example 16. The delivery assembly of any example herein, particularly any one of examples 1 to 12, wherein the perforating guidewire extends proximally from the dilator.

Example 17. The delivery assembly of any example herein, particularly any one of examples 1 to 12, wherein the perforating guidewire is attached to the delivery guidewire.

Example 18. The delivery assembly of any example herein, particularly example 16 or 17, wherein the dilator comprises a dilator channel through which the perforating guidewire extends.

Example 19. The delivery assembly of any example herein, particularly any one of examples 16 to 18, wherein the perforating guidewire extends through the delivery guidewire.

Example 20. The delivery assembly of any example herein, particularly any one of examples 16 to 18, wherein the perforating guidewire and the delivery guidewire are integrally formed.

Example 21. The delivery assembly of any example herein, particularly example 20, wherein the dilator is integrally formed with the delivery guidewire and the perforating guidewire.

Example 22. The delivery assembly of any example herein, particularly any one of examples 16 to 21, further comprising an RF energy source coupled to the perforating guidewire and configured to provide RF energy to the guidewire tip.

Example 23. The delivery assembly of any example herein, particularly any one of examples 1 to 4, wherein a diameter defined by the delivery guidewire is greater than a diameter defined by the perforating guidewire.

Example 24. The delivery assembly of any example herein, particularly any one of examples 1 to 23, wherein the guidewire tip is a sharp tip.

Example 25. The delivery assembly of any example herein, particularly any one of examples 1 to 24, wherein the material from which the dilator is formed has a hardness higher than 80 Shore D.

Example 26. The delivery assembly of any example herein, particularly example 25, wherein the material from which the delivery nosecone is formed has a hardness in a range from 50 Shore A to 80 Shore D.

Example 27. The delivery assembly of any example herein, particularly example 25, wherein the material from which the delivery nosecone is formed has a hardness in a range from 60 Shore A to 100 Shore A.

Example 28. The delivery assembly of any example herein, particularly any one of examples 1 to 27, wherein the delivery guidewire is axially movable with respect to the delivery nosecone.

Example 29. The delivery assembly of any example herein, particularly any one of examples 1 to 28, wherein the perforating guidewire is configured to pierce through a target tissue so as to form a pilot puncture in the tissue.

Example 30. The delivery assembly of any example herein, particularly example 29, wherein the dilator is configured to expand the pilot puncture when passed through the pilot puncture.

Example 31. The delivery assembly of any example herein, particularly any one of examples 1 to 28, further comprising a prosthetic valve comprising a frame movable between a radially compressed and a radially expanded configuration.

Example 32. The delivery assembly of any example herein, particularly example 31, wherein the delivery apparatus further comprises: a handle; a balloon catheter extending from the handle, the balloon catheter defining a balloon catheter lumen; and a balloon mounted on the balloon catheter and in fluid communication with the balloon catheter lumen, the balloon configured to transition between deflated and inflated states thereof.

Example 33. The delivery assembly of any example herein, particularly example 32, wherein the perforating guidewire is configured to pierce through a host leaflet of a host valvular structure so as to form a pilot puncture in the host leaflet.

Example 34. The delivery assembly of any example herein, particularly example 33, wherein the dilator is configured to pass through the pilot puncture and expand the pilot puncture to form a leaflet opening.

Example 35. The delivery assembly of any example herein, particularly example 34, wherein, when the prosthetic valve is disposed around the balloon and positioned within a host valvular structure, inflation of the balloon expands the prosthetic valve to implant the prosthetic valve in the host valvular structure.

Example 36. The delivery assembly of any example herein, particularly any one of examples 33 to 35, wherein the host valvular structure is a native valvular structure of native heart valve.

Example 37. The delivery assembly of any example herein, particularly any one of examples 33 to 35, wherein the host valvular structure is a valvular structure of previously implanted prosthetic valve that is implanted within a native heart valve.

Example 38. The delivery assembly of any example herein, particularly any one of examples 32 to 37, wherein the delivery apparatus further comprises a push shaft configured to push the prosthetic valve from a position proximal to the balloon toward the balloon.

Example 39. The delivery assembly of any example herein, particularly any one of examples 1 to 38, wherein the delivery apparatus is sterilized.

Example 40. A method of forming an opening in a target tissue, the method comprising: advancing a tissue perforation assembly that comprises a dilator distal to a delivery nosecone, to a target tissue, wherein the tissue perforation assembly further comprises: a perforating guidewire attached to the dilator and extending distally therefrom, and a delivery guidewire attached to the dilator and extending proximally therefrom and through a nosecone channel of the delivery nosecone; forming, with the perforating guidewire, a pilot puncture within the target tissue; and advancing the dilator through the pilot puncture to expand the pilot puncture, to form a tissue opening within the target tissue.

Example 41. The method of any example herein, particularly example 40, further comprising, prior to forming the pilot puncture, positioning the perforating guidewire adjacent the target tissue.

Example 42. The method of any example herein, particularly example 40 or 41, wherein the forming the pilot puncture comprises translating the perforating guidewire in a distal direction relative to the delivery nosecone to pierce the target tissue to form the pilot puncture.

Example 43. The method of any example herein, particularly any one of examples 40 to 42, wherein the perforating guidewire extends proximally from the dilator.

Example 44. The method of any example herein, particularly any one of examples 40 to 42, the perforating guidewire is attached to the delivery guidewire.

Example 45. The method of any example herein, particularly example 43 or 44, wherein the perforating guidewire and the delivery guidewire are integrally formed.

Example 46. The method of any example herein, particularly example 45, wherein the dilator is integrally formed with the delivery guidewire and the perforating guidewire.

Example 47. The method of any example herein, particularly any one of examples 43 to 46, wherein the forming the pilot puncture comprises applying RF energy to a tip of the guidewire.

Example 48. The method of any example herein, particularly any one of examples 40 to 47, wherein the dilator is formed from a stiffer material than that of the delivery nosecone.

Example 49. The method of any example herein, particularly any one of examples 40 to 48, further comprising, after the advancing the dilator to form the tissue opening, advancing the delivery nosecone through the tissue opening to further expand the tissue opening.

Example 50. The method of any example herein, particularly any one of examples 40 to 49, wherein the advancing the tissue perforation assembly to the target tissue comprises positioning the dilator in proximity to the target tissue.

Example 51. The method of any example herein, particularly any one of examples 40 to 50, wherein the target tissue is the interatrial septum.

Example 52. The method of any example herein, particularly any one of examples 40 to 50, wherein the target tissue is a host leaflet of a host valvular structure.

Example 53. The method of any example herein, particularly example 52, wherein the host valvular structure is a native valvular structure of native heart valve.

Example 54. The method of any example herein, particularly example 52, wherein the host valvular structure is a valvular structure of previously implanted prosthetic valve that is implanted within a native heart valve.

Example 55. The method of any example herein, particularly any one of examples 52 to 54, wherein the forming the pilot puncture comprises forming a first pilot puncture in a first host leaflet, wherein the advancing the dilator to form the tissue opening comprises advancing the dilator through the first pilot puncture to form a first leaflet opening in the first host leaflet, and wherein, subsequent to forming the first leaflet opening, the method further comprises: retracting the tissue perforating assembly from the first host leaflet; forming, with the perforating guidewire, a second pilot puncture within a second host leaflet; and advancing the dilator through the second pilot puncture to expand the second pilot puncture, to form a second tissue opening within the second host leaflet.

Example 56. The method of any example herein, particularly any one of examples 40 to 51, wherein the advancing the tissue perforation assembly comprises advancing a delivery assembly that comprises a delivery apparatus carrying a prosthetic valve in a radially compressed state, wherein the delivery apparatus comprises the tissue perforation assembly and a balloon mounted on a balloon catheter.

Example 57. The method of any example herein, particularly example 56, wherein the delivery apparatus further comprises a nosecone shaft attached to the delivery nosecone and extending proximally therefrom, the nosecone shaft defining a nosecone shaft lumen sized to allow axial passage of the delivery guidewire therethrough.

Example 58. The method of any example herein, particularly example 57, wherein the nosecone shaft extends through the balloon catheter.

Example 59. The method of any example herein, particularly any one of examples 56 to 58, wherein the target tissue is a host leaflet of a host valvular structure, and wherein the tissue opening is a leaflet opening.

Example 60. The method of any example herein, particularly example 59, further comprising positioning the balloon in a deflated state thereof, along with the prosthetic valve disposed in a compressed state over the balloon, inside the host valvular structure.

Example 61. The method of any example herein, particularly example 60, further comprising inflating the balloon so as to radially expand the prosthetic valve.

Example 62. The method of any example herein, particularly example 61, wherein the positioning the balloon inside the host valvular structure comprises positioning the balloon between host leaflets of the host valvular structure.

Example 63. The method of any example herein, particularly example 62, further comprising, prior to positioning the balloon, proximally retracting the tissue perforation assembly out of the leaflet opening.

Example 64. The method of any example herein, particularly example 61, wherein the positioning the balloon inside the host valvular structure comprises positioning the balloon inside the leaflet opening.

Example 65. The method of any example herein, particularly example 64, wherein the inflating the balloon to radially expand the prosthetic valve increases the size of the leaflet opening.

Example 66. The method of any example herein, particularly example 64 or 65, wherein the inflating the balloon to radially expand the prosthetic valve tears the host leaflet.

Example 67. The method of any example herein, particularly any one of examples 61 to 66, wherein the inflating the balloon to radially expand the prosthetic valve modifies the host leaflet such that the host leaflet does not obstruct a cell opening of a frame of the prosthetic valve.

Example 68. The method of any example herein, particularly any one of examples 61 to 67, wherein the inflating the balloon to radially expand the prosthetic valve moves the host leaflet to a location upstream of a downstream edge of an outer skirt of the prosthetic valve.

Example 69. The method of any example herein, particularly any one of examples 61 to 68, further comprising, prior to positioning the balloon inside the host valvular structure, distally pushing the prosthetic valve, by a push shaft of the delivery apparatus, towards and over the balloon.

Example 70. The method of any example herein, particularly example 69, wherein the positioning the balloon comprises keeping the push shaft in close proximity to a proximal end of the prosthetic valve, so as to provide a counterforce to prevent the prosthetic valve from proximally slipping from the balloon.

Example 71. The method of any example herein, particularly any one of examples 61 to 70, wherein the inflating the balloon comprises providing inflation fluid into the balloon via a lumen of the balloon catheter.

Example 72. The method of any example herein, particularly any one of examples 61 to 70, wherein the host valvular structure is a native valvular structure of native heart valve.

Example 73. The method of any example herein, particularly any one of examples 61 to 71, wherein the host valvular structure is a valvular structure of previously implanted prosthetic valve that is implanted within a native heart valve.

Example 74. The method of any example herein, particularly any one of examples 61 to 73, wherein the forming the pilot puncture comprises forming a first pilot puncture in a first host leaflet, wherein the advancing the dilator to form the leaflet opening comprises advancing the dilator through the first pilot puncture to form a first leaflet opening in the first host leaflet, and wherein, subsequent to forming the first leaflet opening, the method further comprises: retracting the tissue perforating assembly from the first host leaflet; forming, with the perforating guidewire, a second pilot puncture within a second host leaflet; and advancing the dilator through the second pilot puncture to expand the second pilot puncture, to form a second leaflet opening within the second host leaflet.

Example 75. The method of any example herein, particularly example 74, wherein the positioning the balloon inside the host valvular structure comprises positioning the balloon inside the second leaflet opening.

Example 76. The method of any example herein, particularly any one of examples 61 to 75, further comprising, subsequent to inflating the balloon to radially expand the prosthetic valve, deflating the balloon and retrieving the delivery apparatus.

Example 77. A tissue perforation assembly comprising: a delivery nosecone comprising: a nosecone channel; and a nosecone distal tapering portion extending from a nosecone distal end to a nosecone tapering portion proximal end; a dilator distal to the delivery nosecone, the dilator comprising a dilator tapering portion extending from a dilator tapering portion distal end and a dilator tapering portion proximal end; a perforating guidewire attached to the dilator and extending distally from the dilator tapering portion distal end to a guidewire tip; and a delivery guidewire attached to the dilator and extending proximally from the dilator tapering portion proximal end; wherein the perforating guidewire is configured to pierce through a target tissue so as to form a pilot puncture in the tissue; wherein the dilator is formed from a stiffer material than that of the delivery nosecone; and wherein the dilator is configured to expand the pilot puncture when passed through the pilot puncture.

Example 78. The tissue perforation assembly of any example herein, particularly example 77, further comprising a nosecone shaft attached to the delivery nosecone and extending proximally therefrom, the nosecone shaft defining a nosecone shaft lumen sized to allow axial passage of the delivery guidewire therethrough.

Example 79. The tissue perforation assembly of any example herein, particularly example 77 or 78, wherein a diameter defined by the dilator tapering portion distal end is not greater than 120% of a diameter defined by the perforating guidewire.

Example 80. The tissue perforation assembly of any example herein, particularly example 79, wherein the diameter of the dilator tapering portion distal end is not greater than 110% of the diameter of the perforating guidewire.

Example 81. The tissue perforation assembly of any example herein, particularly any one of examples 77 to 80, wherein a diameter defined by the dilator tapering portion proximal end is equal to or greater than a diameter defined by the nosecone distal end.

Example 82. The tissue perforation assembly of any example herein, particularly example 82, wherein the diameter of the dilator tapering portion proximal end is not greater than 120% of the diameter of the nosecone distal end.

Example 83. The tissue perforation assembly of any example herein, particularly example 81, wherein the diameter of the dilator tapering portion proximal end is not greater than 110% of the diameter of the nosecone distal end.

Example 84. The tissue perforation assembly of any example herein, particularly any one of examples 81 to 83, wherein a diameter defined by the nosecone tapering portion proximal end is greater than the diameter of the dilator tapering portion proximal end.

Example 85. The tissue perforation assembly of any example herein, particularly any one of examples 77 to 84, wherein the perforating guidewire and the delivery guidewire are axially spaced from each other.

Example 86. The tissue perforation assembly of any example herein, particularly example 85, wherein the dilator comprises a distal socket to which the perforating guidewire is attached.

Example 87. The tissue perforation assembly of any example herein, particularly example 85 or 86, wherein the dilator comprises a proximal socket to which the delivery guidewire is attached.

Example 88. The tissue perforation assembly of any example herein, particularly any one of examples 77 to 84, wherein the perforating guidewire extends proximally from the dilator.

Example 89. The tissue perforation assembly of any example herein, particularly any one of examples 77 to 84, wherein the perforating guidewire is attached to the delivery guidewire.

Example 90. The tissue perforation assembly of any example herein, particularly example 88 or 89, wherein the dilator comprises a dilator channel through which the perforating guidewire extends.

Example 91. The tissue perforation assembly of any example herein, particularly any one of examples 88 to 90 wherein the perforating guidewire extends through the delivery guidewire.

Example 92. The tissue perforation assembly of any example herein, particularly any one of examples 88 to 90, wherein the perforating guidewire and the delivery guidewire are integrally formed.

Example 93. The tissue perforation assembly of any example herein, particularly example 92, wherein the dilator is integrally formed with the delivery guidewire and the perforating guidewire.

Example 94. The tissue perforation assembly of any example herein, particularly any one of examples 88 to 93, further comprising an RF energy source coupled to the perforating guidewire and configured to provide RF energy to the guidewire tip.

Example 95. The tissue perforation assembly of any example herein, particularly example 77, wherein a diameter defined by the delivery guidewire is greater than a diameter defined by the perforating guidewire.

Example 96. The tissue perforation assembly of any example herein, particularly any one of examples 77 to 95, wherein the guidewire tip is a sharp tip.

Example 97. The tissue perforation assembly of any example herein, particularly any one of examples 77 to 96, wherein the material from which the dilator is formed has a hardness higher than 80 Shore D.

Example 98. The tissue perforation assembly of any example herein, particularly example 97, wherein the material from which the delivery nosecone is formed has a hardness in a range from 50 Shore A to 80 Shore D.

Example 99. The tissue perforation assembly of any example herein, particularly example 97, wherein the material from which the delivery nosecone is formed has a hardness in a range from 60 Shore A to 100 Shore A.

Example 100. The tissue perforation assembly of any example herein, particularly any one of examples 77 to 99, wherein the delivery guidewire is axially movable with respect to the delivery nosecone.

Example 101. A delivery assembly comprising: a guest prosthetic valve comprising a frame movable between a radially compressed and a radially expanded configuration; and a delivery apparatus comprising: a handle; a balloon catheter extending from the handle, the balloon catheter defining a balloon catheter lumen; a balloon catheter extending from the handle, the balloon catheter defining a balloon catheter lumen; a balloon mounted on the balloon catheter and in fluid communication with the balloon catheter lumen, the balloon configured to transition between deflated and inflated states thereof; a tissue perforation assembly comprising: a delivery nosecone distal to the balloon, the delivery nosecone comprising a nosecone channel and a nosecone distal tapering portion extending from a nosecone distal end to a nosecone tapering portion proximal end; a dilator distal to the delivery nosecone, the dilator comprising a dilator tapering portion extending from a dilator tapering portion distal end and a dilator tapering portion proximal end; a perforating guidewire attached to the dilator and extending distally from the dilator tapering portion distal end to a guidewire tip; and a delivery guidewire attached to the dilator and extending proximally from the dilator tapering portion proximal end; wherein the perforating guidewire is configured to pierce through a host leaflet of a host valvular structure so as to form a pilot puncture in the host leaflet; wherein the dilator is configured to pass through the pilot puncture and expand the pilot puncture to form a leaflet opening; and wherein, when the guest prosthetic valve is disposed around the balloon and positioned within a host valvular structure, inflation of the balloon expands the guest prosthetic valve to implant the guest prosthetic valve in the host valvular structure.

+Example 102. The delivery assembly of any example herein, particularly example 101, wherein the delivery apparatus further comprises a push shaft configured to push the guest prosthetic valve from a position proximal to the balloon toward the balloon.

Example 103. The delivery assembly of any example herein, particularly any one of examples 101 to 102, wherein the host valvular structure is a native valvular structure of native heart valve.

Example 104. The delivery assembly of any example herein, particularly any one of examples 101 to 102, wherein the host valvular structure is a valvular structure of previously implanted prosthetic valve that is implanted within a native heart valve.

Example 105. The delivery assembly of any example herein, particularly any one of examples 101 to 103, wherein the delivery apparatus is sterilized.

Example 106. A method of implanting a guest prosthetic valve within a host valvular structure, the method comprising: advancing a delivery assembly that comprises a delivery apparatus carrying a guest prosthetic valve in a radially compressed state, to a host valvular structure, wherein the delivery apparatus comprises a balloon mounted on a balloon catheter and a tissue perforation assembly which comprises: a delivery nosecone distal to the balloon, a dilator distal to the delivery nosecone, a perforating guidewire attached to the dilator and extending distally therefrom, and a delivery guidewire attached to the dilator and extending proximally therefrom and through a nosecone channel of the delivery nosecone; forming, with the perforating guidewire, a pilot puncture within a host leaflet of the host valvular structure; advancing the dilator through the pilot puncture to expand the pilot puncture, to form a leaflet opening within the host leaflet; positioning the balloon in a deflated state thereof, along with the guest prosthetic valve disposed in a compressed state over the balloon, inside the host valvular structure; and inflating the balloon so as to radially expand the guest prosthetic valve.

Example 107. The method of any example herein, particularly example 106, further comprising, prior to forming the pilot puncture, positioning the perforating guidewire adjacent the target tissue.

Example 108. The method of any example herein, particularly example 106 or 107, wherein the forming the pilot puncture comprises translating the perforating guidewire in a distal direction relative to the delivery nosecone to pierce the target tissue to form the pilot puncture.

Example 109. The method of any example herein, particularly any one of examples 106 to 108, wherein the perforating guidewire extends proximally from the dilator.

Example 110. The method of any example herein, particularly any one of examples 106 to 108, the perforating guidewire is attached to the delivery guidewire.

Example 111. The method of any example herein, particularly example 109 or 110, wherein the perforating guidewire and the delivery guidewire are integrally formed.

Example 112. The method of any example herein, particularly example 111, wherein the dilator is integrally formed with the delivery guidewire and the perforating guidewire.

Example 113. The method of any example herein, particularly any one of examples 109 to 112, wherein the forming the pilot puncture comprises applying RF energy to a tip of the guidewire.

Example 114. The method of any example herein, particularly any one of examples 106 to 113, wherein the dilator is formed from a stiffer material than that of the delivery nosecone.

Example 115. The method of any example herein, particularly any one of examples 106 to 114, further comprising, after the advancing the dilator to form the leaflet opening, advancing the delivery nosecone through the leaflet opening to further expand the leaflet opening.

Example 116. The method of any example herein, particularly any one of examples 106 to 115, wherein the advancing the tissue perforation assembly to the host valvular structure comprises positioning the dilator in proximity to the host leaflet.

Example 117. The method of any example herein, particularly any one of examples 106 to 116, wherein the positioning the balloon inside the host valvular structure comprises positioning the balloon between host leaflets of the host valvular structure.

Example 118. The method of any example herein, particularly example 117, further comprising, prior to positioning the outer balloon, proximally retracting the tissue perforation assembly out of the leaflet opening.

Example 119. The method of any example herein, particularly any one of examples 106 to 116, wherein the positioning the balloon inside the host valvular structure comprises positioning the balloon inside the leaflet opening.

Example 120. The method of any example herein, particularly example 119, wherein the inflating the balloon to radially expand the guest prosthetic valve increases the size of the leaflet opening.

Example 121. The method of any example herein, particularly example 119 or 120, wherein the inflating the balloon to radially expand the guest prosthetic valve tears the host leaflet.

Example 122. The method of any example herein, particularly any one of examples 106 to 121, wherein the inflating the balloon to radially expand the guest prosthetic valve modifies the host leaflet such that the host leaflet does not obstruct a cell opening of a frame of the guest prosthetic valve.

Example 123. The method of any example herein, particularly any one of examples 106 to 122, wherein the inflating the balloon to radially expand the guest prosthetic valve moves the host leaflet to a location upstream of a downstream edge of an outer skirt of the guest prosthetic valve.

Example 124. The method of any example herein, particularly any one of examples 106 to 123, wherein the delivery nosecone shaft extends through the balloon catheter.

Example 125. The method of any example herein, particularly any one of examples 106 to 124, further comprising, prior to positioning the balloon inside the host valvular structure, distally pushing the guest prosthetic valve, by a push shaft of the delivery apparatus, towards and over the balloon.

Example 126. The method of any example herein, particularly example 125, wherein the positioning the balloon comprises keeping the push shaft in close proximity to a proximal end of the guest prosthetic valve, so as to provide a counterforce to prevent the guest prosthetic valve from proximally slipping from the balloon.

Example 127. The method of any example herein, particularly any one of examples 106 to 126, wherein the inflating the balloon comprises providing inflation fluid into the balloon via a lumen of the balloon catheter.

Example 128. The method of any example herein, particularly any one of examples 106 to 127, wherein the host valvular structure is a native valvular structure of native heart valve.

Example 129. The method of any example herein, particularly any one of examples 106 to 127, wherein the host valvular structure is a valvular structure of previously implanted prosthetic valve that is implanted within a native heart valve.

Example 130. The method of any example herein, particularly any one of examples 106 to 129, wherein the forming the pilot puncture comprises forming a first pilot puncture in a first host leaflet, wherein the advancing the dilator to form the tissue opening comprises advancing the dilator through the first pilot puncture to form a first leaflet opening in the first host leaflet, and wherein, subsequent to forming the first leaflet opening, the method further comprises: retracting the tissue perforating assembly from the first host leaflet; forming, with the perforating guidewire, a second pilot puncture within a second host leaflet; and advancing the dilator through the second pilot puncture to expand the second pilot puncture, to form a second tissue opening within the second host leaflet.

Example 131. The method of any example herein, particularly example 130, wherein the positioning the balloon inside the host valvular structure comprises positioning the balloon inside the second leaflet opening.

Example 132. The method of any example herein, particularly any one of examples 106 to 131, further comprising, subsequent to inflating the balloon to radially expand the guest prosthetic valve, deflating the balloon and retrieving the delivery apparatus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery assembly, comprising:
a delivery apparatus comprising a tissue perforation assembly, the tissue perforation assembly comprising:
a delivery nosecone comprising:
a nosecone channel; and
a nosecone distal tapering portion between a nosecone distal end and a nosecone tapering portion proximal end;
a dilator comprising a dilator tapering portion between a dilator tapering portion distal end and a dilator tapering portion proximal end;
a perforating guidewire extending distally from the dilator tapering portion distal end to a guidewire tip; and
a delivery guidewire extending proximally from the dilator tapering portion proximal end.

2. The delivery assembly of claim 1, wherein the dilator is distal to the delivery nosecone.

3. The delivery assembly of claim 1, wherein the perforating guidewire is attached to the dilator.

4. The delivery assembly of claim 1, wherein the delivery guidewire is attached to the dilator.

5. The delivery assembly of claim 1, wherein the dilator is formed from a stiffer material than that of the delivery nosecone.

6. The delivery assembly of claim 1, wherein the tissue perforation assembly further comprises a nosecone shaft attached to the delivery nosecone and extending proximally therefrom, the nosecone shaft defining a nosecone shaft lumen sized to allow axial passage of the delivery guidewire therethrough.

7. The delivery assembly of claim 1, wherein a diameter defined by the dilator tapering portion proximal end is equal to or greater than a diameter defined by the nosecone distal end.

8. The delivery assembly of claim 7, wherein the diameter of the dilator tapering portion proximal end is not greater than 120% of the diameter of the nosecone distal end.

9. The delivery assembly of claim 1, wherein the perforating guidewire and the delivery guidewire are integrally formed.

10. The delivery assembly of claim 9, wherein the dilator is integrally formed with the delivery guidewire and the perforating guidewire.

11. The delivery assembly of claim 1, wherein the material from which the dilator is formed has a hardness higher than 80 Shore D.

12. The delivery assembly of claim 11, wherein the material from which the delivery nosecone is formed has a hardness in a range from 50 Shore A to 80 Shore D.

13. The delivery assembly of claim 1, wherein the perforating guidewire is configured to pierce through a target tissue so as to form a pilot puncture in the tissue.

14. A method of forming an opening in a target tissue, the method comprising:
advancing a tissue perforation assembly that comprises a dilator distal to a delivery nosecone, to a target tissue, wherein the tissue perforation assembly further comprises: a perforating guidewire attached to the dilator and extending distally therefrom, and a delivery guidewire attached to the dilator and extending proximally therefrom and through a nosecone channel of the delivery nosecone;
forming, with the perforating guidewire, a pilot puncture within the target tissue; and
advancing the dilator through the pilot puncture to expand the pilot puncture, to form a tissue opening within the target tissue.

15. The method of claim 14, wherein the forming the pilot puncture comprises translating the perforating guidewire in a distal direction relative to the delivery nosecone to pierce the target tissue to form the pilot puncture.

16. The method of claim 14, wherein the forming the pilot puncture comprises applying RF energy to a tip of the guidewire.

17. The method of claim 14, wherein the dilator is formed from a stiffer material than that of the delivery nosecone.

18. The method of claim 14, further comprising, after the advancing the dilator to form the tissue opening, advancing the delivery nosecone through the tissue opening to further expand the tissue opening.

19. The method of claim 14, wherein the advancing the tissue perforation assembly comprises advancing a delivery assembly that comprises a delivery apparatus carrying a prosthetic valve in a radially compressed state, wherein the delivery apparatus comprises the tissue perforation assembly and a balloon mounted on a balloon catheter.

20. The method of claim 19, wherein the target tissue is a host leaflet of a host valvular structure, and wherein the tissue opening is a leaflet opening.

21. The method of claim 20, further comprising positioning the balloon in a deflated state thereof, along with the prosthetic valve disposed in a compressed state over the balloon, inside the host valvular structure.

22. The method of claim 21, further comprising inflating the balloon so as to radially expand the prosthetic valve.
